(19) United States
(12) Patent Application Publication
(10) Pub. No.: US 2011/0165092 A1
(43) Pub. Date: Jul. 7, 2011

(54) COSMETIC AND/OR DERMATOLOGICAL COMPOSITION

(75) Inventors: Pierre Cau, Puyricard (FR); Patrice Bourgeois, Vitrolles (FR); Vincent Bonniol, Marseille (FR); Nicolas Levy, Marseille (FR)

(73) Assignee: Universite de la Mediterranee, Aix-Marseille II, Marseillle (FR)

(21) Appl. No.: 12/811,622

(22) PCT Filed: Dec. 31, 2008
(86) PCT No.: PCT/FR2008/001843

§ 371 Date: Mar. 16, 2011

(30) Foreign Application Priority Data
Jan. 3, 2008 (FR) 0850019

(51) Int. Cl. A61K 31/675; A61K 31/225; A61K 8/37; A61K 8/55; A61K 31/663; A61K 31/351; A61K 8/49; A61Q 17/04; A61K 9/12; A61Q 19/08; A61Q 7/00; A61P 17/14; A61P 17/00

(52) U.S. Cl. 424/43; 514/548; 514/94; 514/108; 514/460; 424/59; 424/70.9

(57) ABSTRACT

The subject matter of the invention is a cosmetic and/or dermatological composition for use in the treatment of skin and/or hair disorders. More particularly, the invention relates to a cosmetic and/or dermatological composition comprising an inhibitor of hydroxymethylglutaryl-coenzyme A (HMG-CoA) reductase, an inhibitor of farnesyl pyrophosphate synthase, or an inhibitor of one of the physiologically acceptable salts thereof and a cosmetic and/or dermatological product. The present invention finds, for example, a very advantageous use in the treatment of the effects of early ageing, in particular in terms of the skin and the hair system.

### TECHNICAL FIELD

This invention relates to a cosmetic and/or dermatological composition for use in the treatment of skin and hair disorders.

In the following description, the references between parentheses (X) refer to the list of references at the end of the examples.

### PRIOR ART

The nucleus of eukaryotic cells is defined by a double membrane perforated with pores, the nuclear envelope, which controls the molecular exchanges between the two nuclear and cytoplasmic compartments. This envelope partially isolates the contents of the nucleus, i.e., the genetic material and all of the enzymatic machinery required for the functions of the nuclear genome.

The nuclear envelope consists of 2 concentric membranes, the outer membrane, continuous with the endoplasmic reticulum, and the inner membrane. The latter is bordered on the inner face thereof by a dense fibrillar network call the nuclear lamina. This is a protein network consisting substantially of lamin polymers and associated proteins. In vertebrates, two sub-classes of lamins are identified: type A lamins (lamins A and C), and type B lamins (lamins B1, B2 and B3), all of which participate in the development of the lamina. The latter is held in place by association with other proteins attached to the inner membrane of the nuclear envelope (for an overview, see Gruenbaum et al., 2005 (19)).

Lamins are filament-like proteins belonging to the family of intermediate filaments (type V) all of which have common structure: a short N-terminal globular segment (head) separated from another C-terminal globular segment (tail) by a long central domain divided into several alpha (rod domain) helixes. The globular tail contains, in particular, a nuclear localization signal (NLS) enabling targeting to the nucleus after synthesis. The central domain enables the association of two parallel lamin molecules and the organisation thereof into filaments, via association of the “head-to-tail” dimers. This structure gives them very strong mechanical properties.

Only the A lamin and the B lamins undergo maturation after the synthesis of a precursor (for an overview, see Gruenbaum et al., 2000 (20)). Lamin C is synthesized directly in the mature form thereof.

The precursor of lamin A and lamins B terminates in a characteristic CaaX motif (C is a cysteine, a an amino acid with an uncharged aliphatic chain and X is any amino acid, which, in this case is methionine (for an overview, see Lévy & Cau 2003 (29)).

The C-terminal CaaX motif enables the attachment of a fatty acid (generally a fatty acid at C15, farnesyl), owing to a farnesyl transferase. This prenylation (the farnesyl unit derived from a basic aliphatic unit at C5 called isoprene) enables the prelamins to be inserted into the membrane of the endoplasmic reticulum after the synthesis of same in the cytosol. They undergo the effects therein of an endoprotease, which is itself inserted into the envelope membrane of the reticulum and the active site of which is cytosolic. The specific endoprotease of prelamin A is Face1 (or ZMPSTE24, Zinc Metallo-Protease STE24 homologue of yeast), while Face2 (or Rce1, ras-converting enzyme) is specific to the prelamins B. These enzymes catalyze the hydrolyse of the peptide bond between the cysteine and the following (aliphatic) amino acid, shortening the prelamins of 3 amino acids. The carboxyl end of the farnesylated cysteine is next recognized by an isoprenylcysteine-carboxymethyl transferase (ICMT), which attaches a methyl group thereto via esterification.

The maturation of the prelamin A continues only with a second endoproteolytic cleavage via Face1, which releases a farnesyl-peptide of 15 amino acids and the mature lamin A. This lamin A, which no longer contains the fatty acid, becomes soluble, is imported into the nucleus owing to the nuclear localization signal thereof, and is localized in the nuclear lamina itself as well as in the rest of the nuclear compartment, thereby forming a true nuclear skeleton (Gruenbaum et al., 2005 (19)). On the other hand, at the C-terminal end, the cysteine of the mature lamin B is always farnesylated and methyl-esterified. It therefore remains inserted into the envelope membrane of the reticulum, and then into the nucleoplasmic face of the nuclear envelope, hence its exclusive localization at the nuclear lamina, beneath the inner membrane of the nuclear envelope wherein it is anchored.

The term prenylation is understood to mean the attachment to the thiol group of a cysteine of either a farnesyl chain of 15 carbon atoms, which is then referred to as farnesylation, or of a geranyl-geranyl chain of 20 carbon atoms, which is then referred to as geranyl-geranylation (Reid et al., 2004 (39)), or else of any other isoprene derivative.

The farnesylation step, which is catalyzed by the farnesyltransferase (FTase), which recognizes the C-terminal consensus sequence (CaaX), preferably attaches a farnesyl group onto the cysteine residue of the unit.

Geranylgeranylation is the attachment by the geranylgeranyl-transferase (GGTase) of a geranylgeranyl group onto the cysteine residue of the unit.

The fatty acids are derived from the biosynthesis pathway, which is used by the cells to manufacture cholesterol, steroids, the heme in haemoglobin and ubiquinones (Hampton et al., 1996 (20)).

The family of prenylated proteins comprises approximately 300 members in the human genome, the majority of which is identifiable by the CaaX C-terminal motif (Reid et al., 2004 (39)). The proteins of the Ras, Rho, Rab families (Leung et al., 2006 (28)), some proteins providing in import function towards the mitochondria (HDJ2), and some proteins mitotic proteins (CENPE, CENPF) are in particular prenylated (Winter-Vann & Casey 2005 (51)). In general if, in the CaaX motif, X is a serine, a methionine, a cysteine, an alanine or a glutamate, the preferably grafted isoprenoid is farnesyl. If X is a leucine, recognition of the CaaL motif will preferably occur via the GGTase, which will catalyze the transfer of a geranylgeranyl group (Basso et al., 2006 (1)). It is probable that other isoprene derivative groups can also be attached to this cysteine, even though that is not described in literature.

Three lamin genes exist in man. The LMNA gene situated at 1q21.2-q21.3 (Wydner et al., 1996 (52)), yields lamins A and C by way of alternative splicing. The LMNA gene consists of 12 exons. The start of exon 1 codes the N-terminal globular end common to lamins A and C; the end of exon 1 and up to the start of exon 7 codes the central helicoidal portion; finally, the other exons code the C-terminal globular end (Lévy & Cau 2003 (29)).

In fact, the gene codes differently for 4 spliced products, of which the lamins C and the prelamin A are the 2 primary ones (Lin & Worman 1993 (31)). The differential production of lamins A and C is carried out by the use of an alternative splicing site of exon 10 of the pre-messenger, whereby lamin C is coded by exons 1 to 10 and lamin A is coded by exons 1 to 9, the first 90 base pairs of exon 10, and exons 11 and 12 (lamin A-specific).

Consequently, the prelamin A and lamin C peptides are identical in the first 566 amino acids, the C-terminal ends of lamins C and of prelamin A then contain 6 and 98 specific amino acids, respectively.

The type B lamins include three different proteins (Shelton et al., 1981 (43)): lamins B1, B2 (the two isoforms most represented) and B3. The LMNB1 gene is situated at 5q23.3-q31.1 and comprises 11 exons coding lamin B1 (Lin & Worman 1995 (30)). The LMNB2 gene is located at 19p13.3 and codes for lamins B1 and B3 via an alternative splicing mechanism (Biamonti et al., 1992 (2)).

Type B lamins are expressed consecutively in all cells from the first stages of development, whereas type A lamins are generally absent in embryonic stem cells (Stewart et al., 1987 (45)) and are expressed in all differentiated somatic cells. Their expression is subject to regulations, depending on the tissue and over the course of life (Duque et al., 2006 (9)). It appears that their expression is not necessary, since mice in which the expression of lamin A was specifically blocked, but which nonetheless express lamin C and the other lamins, do not have any apparent phenotype (Fong et al., 2006 (14)).

Lamins interact with a very high number of protein partners having very diverse functions; they are therefore involved in a large number of nuclear processes, including DNA replication and repair, transcription and splicing control, organisation of the chromatin structure (for an overview, see Shumaker et al., 2003 (44), Zastrow et al., 2004 (54), Hutchinson et al., 2004 (26), Gruenbaum et al., 2005 (19)). The alterations of the lamina structure are the source of numerous hereditary human diseases. They are due to mutations of the genes coding the lamins, or of other proteins of the lamina. These diseases have been grouped together under the generic term of laminopathies (Broer et al., 2006 (5), Mattout et al., 2006 (33)). Mutations in the genes of the enzymes responsible for the maturation of the lamins (Face1 in particular) have recently been identified, which give rise to diseases likewise belonging to the group of laminopathies (Navarro et al., 2004 (36) and 2005 (35)).

To date, the only disease in man which is associated with mutations of the LMNB1 or 2 genes is a leukodystrophy caused by a complete duplication of the LMNB1 gene (Padiath et al., 2006 (37)). Doubt remains as to the potential involvement of variations of sequences found in LMNB2 in patients afflicted with Barraquer-Simons syndrome (Hegele et al., 2006 (22)). However, in vitro experiments with RNAi (RNA-interference) (Harborth et al., 2001 (21)), as well as in with the murine model (Vergnes et al., 2004 (50), have shown that type B lamins are essential to cell development and integrity. As a matter of fact, B1 lamin deficiency in mice results in perinatal lethality. In addition, the nuclei of the embryonic fibroblasts of the same LMNB1-deficient mice show remarkable alterations in the nuclear morphology, close to those observed in patients carrying mutations of the LMNA gene. Furthermore, it has recently been shown that the B lamins required for the formation of the spindle during mitosis, which tends to prove that the role thereof is dynamic and multiple over the cell cycle, and not solely restricted to maintaining the architecture of the nucleus (Tsai et al., 2006 (48)). As concerns this latter role, a recent article demonstrates the structural function of the lamins B: cells artificially deprived of lamins B1 have a “floating” nucleus in the cell, which rotates about itself (Liu et al., 2007 (45)). The functional redundancy existing between the two lamins B1 and B2 is undoubtedly also a direct reflection of the criticality thereof, which exerts heavy selection pressure and masks the effect of possible mutations in the sequence of the corresponding genes.

The functional alterations of the lamins A/C, which are due to mutations in the LMNA gene, are at the source of at least 15 disorders encompassing very diverse diseases within a clinical spectrum ranging from less serious forms affecting a single tissue in an isolated manner, to lethal systemic forms during the perinatal period.

A number of mutations of the LMNA gene significantly modify the assembly of proteins in the nuclear envelope and disrupt the operation thereof. The morphology of the nuclei is altered in the cells of various tissues: they often have hernias which extrude from the genetic material in the cytoplasm (Goldman et al., 2004 (18)).

The proteins commonly associated with the nuclear envelope, the lamins B, certain proteins of the nuclear pores and the LAP2 proteins are absent from the periphery of these hernias.

These morphological anomalies are followed by functional alterations, which result in cell death. Amongst all of the diseases grouped together under the name of laminopathies, only those associated with the abnormal accumulation of a prenylated form of protein are of concern in this invention.

These primarily involve the Hutchinson-Gilford or Progeria syndrome (De Sandre-Giovannoli et al., 2003 (7), Eriksson et al., 2003 (11)), and restrictive dermopathy (Navarro et al., 2004 (36)). In these two syndromes, the physiopathological cause is an accumulation and persistence of immature farnesylated prelamin in the cells of patients.

Restrictive dermopathy, which is lethal around the natal period, is characterised by clinical signs which are nearly all the result of a deficiency in the skin which restricts in utero movements. This disease is very rare. The skin is rigid and tight, it sags in places, thereby causing tears, for example, in the vicinity of the arm pits and neck. The eyelashes, eyebrows and hair on the skin are absent or very sparse. Hydramnios is often present, and the decrease in foetal movement is reported from the 6^{th} month of pregnancy. In terms of the skeletal system, X-rays reveal contractures in all of the joints, “ice-pick” feet, thin, dysplastic and bipartite clavicles, ribbon ribs, long tubular arm bones and demineralisation of the skull. Transmission of the lethal restrictive dermopathy is autosomal recessive.

Mutations of LMNA and ZMPSTE24/Face1 were have been reported for this disease (Navarro et al., 2004 (36)). In both cases, the physiopathological mechanism is the same: the prelamin A cannot mature (no mutation of Face1 or disappearance of the cleavage site by mutation of the prelamin A), and remains farnesylated, and therefore inserted into nuclear membrane. The accumulation and persistence of these abnormal precursors in the cells, which probably prevents the normal interaction of the lamins B and C with their partners, results in the death of the cells and, in the short run, of the patient. It was clearly shown that it is indeed the persistence of the farnesyl group, and not the absence of the mature lamin A, as might first have been thought, which is responsible for cell toxicity (Fong et al., 2004 (16)).

In April 2003, based on an overlapping of the symptoms common to acromandibular dysplasia and to certain diseases resulting in premature ageing, the inventors demonstrated that Progeria, which is the most typical and serious form of premature ageing, results from a mutation of the LMNA gene (De Sandre-Giovannoli et al., 2003 (7)). Children afflicted with this disease, which is also called Hutchinson-Gilford syndrome, suffer from accelerated ageing, up to ten times more rapid than that of a normal individual, and have a life expectancy which does not exceed 13 years. In Europe, one child in approximately six million is affected. The symptoms are ageing of the skin, baldness, reduction in the size of the jaw and problems associated with old age, e.g., stiffness in the joints and cardiovascular disorders. The latter, such as myocardial infarction or atherosclerosis, are often the cause of death.

The responsible mutation, situated in exon 11 of the LMNA gene, activates a cryptic splicing site of the pre-RNAm, resulting in an RNAm with 150 nucleotides deleted (De Sandre-Giovannoli et al., 2003 (7), Eriksson et al., 2003 (11)). This deleted RNAm is converted into an abnormal prelamin A, progerin, which cannot be matured into normal lamin A: the absence of 50 amino acids of exon 11 comprising the recognition site of the protease blocks the 2^{nd} cleavage of the progerin, the C-terminal end of which retains the farnesyl group thereof. It therefore remains inserted into the nucleoplasmic face of the nuclear envelope, which has characteristic alterations, hernias of the nucleoplasm in the cytosol and anomalies in the distribution of the peripheral heterochromatin (Goldman et al., 2004 (18)). Here again, it is the persistence of the farnesyl group, which is also necessary for anchoring the envelope membrane of the reticulum in which some of the enzymes responsible for maturation are located (cleavages, methylation), which is responsible for the cell toxicity of the progerin (Fong et al., 2004 (16)).

These systemic diseases have the particular characteristic of being associated with the premature appearance of signs commonly associated with ageing. Their common physiopathological characteristic is that of generating a prenylated lamin, along with the described consequences.

Two recent studies have shown that a reduction in intranuclear accumulation of the truncated or non-truncated farnesylated prelamin effectively prevents appearance of the cell phenotype. The first one was conducted on the Face1 protease-deficient progeroid murine model (Pendas et al., 2002 (38)). When they are crossed with mice expressing less than half Lamin A (Lmna+/−mice), the effects of the absence of Face1 are lessened (Varela et al., 2005 (49)). The second study shows that the treatment of cells of HGPS patients with a morpholino (antisense oligonucleotides) targeting the cryptic splicing site abolishes the mutant phenotype (Scaffidi & Mistelli 2005 (43)).

Several recent studies (see Scaffidi & Mistelli 2006 (42)) show the involvement of lamin A in the physiological ageing process. In particular, it has been demonstrated that, during physiological ageing, progerin is synthesized by the cells in the absence of any mutation of the LMNA gene, due to the low-noise use of the cryptic splicing site of exon 11. This progerin is localised in the lamina, at the periphery of the cell nucleus. The nucleus of cells of “normal” elderly patients may have hernias characteristic of a laminopathy caused by accidental splicing events, which result in anomalies in the cell functions and are probably at least partially responsible for the ageing thereof.

In the case of in vivo skin, progerin is also synthesized by a sub-population of dermal fibroblasts and keratinocytes, cells in which it accumulates with age. Progerin could therefore be a marker for skin ageing (McClintock et al., 2007 (34)).

It appears that identical molecular mechanisms are, on the one hand, responsible for the signs of premature ageing in individuals afflicted with Progeria and, on the other hand, to a much less extent, involved in the physiological ageing of individuals not carrying mutations.

There are two therapeutic approaches described in the prior art for improving the cell phenotype caused by the pathological production of progerin. The first of these solutions is quite simply to prevent the use of this cryptic splicing site in exon 11 via the spliceosome, while “masking” it by treating with an antisense oligonucleotide (Scaffidi & Misteli 2005 (41)), or with a retrovirus producing an siRNA (Huang et al., 2005 (25)). The results are promising in vitro, but, in this case, it is a matter of “gene” therapy, and the development of a drug based on this approach is inevitably long and complicated, with all of the drawbacks related to vectorization of the ASOs in order to obtain an in vivo effect. The second solution consists of inhibiting the farnesyltransferase, the enzyme which catalyzes the transfer of the farnesyl group to the prelamins using farnesyl pyrophosphate. When such inhibitors (FTI) are used, a “normal” nuclear envelope is only partially restored on HGPS (Progeria) cells in culture, and the survival of RD mice (KO ZMPSTE24) is improved (Glynn & Glover 2005 (17), Capell et al., 2005 (6), Toth et al., 2005 (47), Fong et al., 2006 (15)).

However, blocking the farnesylation can induce a compensatory geranylgeranylation (Bishop et al., 2003 (3), Varela et al., 2008 (55)).

On the other hand, it was recently reported that FTIs cause a stoppage of the cell cycle by blocking the proteasome (Demyanets et al., 2006 (8), Efuet & Keymarsi 2006 (10)). Thus, the treatment undoubtedly causes an accumulation of progerin in the nucleoplasm, which is probably ubiquitinylated and undegraded by the proteasome.

Additionally, recent studies report that the decrease in the farnesylation rate of the progerin in vivo is very low, of the order of 5% (Young et al., 2006 (53)), which is not sufficient to explain the restoration of the nuclear morphology observed in vitro.

Finally, the FTIs are specific to a single one of the protein prenylation pathways, and cannot be anticipated as global inhibitors of post-translational prenylation.

In addition, it is reported that the total absence of one of the enzymes of this pathway, mevalonate kinese, is lethal during childhood (loss-of-function homozygous mutation of the gene coding for this enzyme, a syndrome reported by Hoffmann et al., 2003 (24)).

### DISCLOSURE OF THE INVENTION

After lengthy research, the inventors have shown that the association of an inhibitor of hydroxymethylglutaryl-coenzyme A reductase (family of statins), an inhibitor of farnesyl pyrophosphate synthase (aminobiphosphonate family, NBPs), or of one of the physiologically acceptable salts thereof, is an effective treatment for the pathological or non-pathological situations associated with the accumulation and/or persistence of prenylated proteins in cells, insofar as it acts on the entire protein prenylation pathway, both at C15 and at C20 or in the non-characterised forms. The inventors have further ascertained that the association of an inhibitor of hydroxymethylglutaryl-coenzyme A (HMG-CoA) reductase and an inhibitor of farnesyl pyrophosphate synthase has a synergistic effect in the restoration of the normal phenotype in fibroblasts of patients afflicted with Progeria. The effect of the association is markedly greater than the effect of either of the inhibitors used individually.

Use of the association on cells of patients afflicted with Progeria and in a mouse model reproducing restrictive dermopathy results in an inhibition of protein prenylation, and therefore in the appearance of non-farnesylated prelamin A and in the improvement of the nuclear symptoms (Varela et al., 2008 (24)).

This invention relates therefore to the use of at least one inhibitor of hydroxymethylglutaryl-coenzyme A (HMG-CoA) reductase and at least one inhibitor of farnesyl pyrophosphate synthase, or of one of the physiologically acceptable salts thereof, in the preparation of a cosmetic and/dermatological composition. This composition, for example, can be intended for the treatment of pathological or non-pathological situations associated with the accumulation and/or persistence of prenylated proteins in cells.

In particular, this invention relates to a cosmetic and/or dermatological composition including:
a. at least one inhibitor of hydroxymethylglutaryl-coenzyme A (HMG-CoA) reductase,
b. at least one inhibitor of farnesyl pyrophosphate synthase, and
c. at least one cosmetic and/or dermatological product.

According to another aspect, this invention likewise relates to:
Joint or simultaneous use, i.e., the at least two following products (the at least one inhibitor of hydroxymethylglutaryl-coenzyme A reductase, on the one hand, and the at least one inhibitor of farnesyl pyrophosphate synthase) are administered in a single composition containing same,
Concomitant use, i.e., separately, each of the at least two following products (the at least one inhibitor of hydroxymethylglutaryl-coenzyme A reductase, on the one hand, and the at least one inhibitor of farnesyl pyrophosphate synthase) being administered independently via identical or different routes of administration, the administration of these two products being carried out concomitantly or
Successive use, that is separately, each of the at least two following products (the at least one inhibitor of hydroxymethylglutaryl-coenzyme A reductase, on the one hand, and the at least one inhibitor of farnesyl pyrophosphate synthase) being administered independently via identical or different routes of administration, the administration of these two products being carried out successively, one product after the other,

the use of at least one inhibitor of hydroxymethylglutaryl-coenzyme A (HMG-CoA) reductase, and the at least one inhibitor of farnesyl pyrophosphate synthase in the treatment of skin and/or hair disorders.

Thus, according to this aspect, the aforesaid compounds can be used in a mixture for simultaneous administration or separately for concomitant or successive administration.

For example, the at least one inhibitor of hydroxymethylglutaryl-coenzyme A (HMG-CoA) reductase can be administered first, then the at least one inhibitor of farnesyl pyrophosphate synthase can be administered secondly. In another example, the at least one inhibitor of farnesyl pyrophosphate synthase can be administered first, then the at least one inhibitor of hydroxymethylglutaryl-coenzyme A (HMG-CoA) reductase can be administered secondly.

According to a third example, the at least one inhibitor of farnesyl pyrophosphate synthase, on the one hand, and the at least one inhibitor of hydroxymethylglutaryl-coenzyme A (HMG-CoA) reductase, on the other hand, are administered at the same time but, for example via different routes of administration, e.g., chosen from amongst oral administration, topical administration or by injection.

Irrespective of the embodiment of this invention, in particular in the aforesaid aspects,

For example, the composition used in this invention can be prepared extratemporaneously at the moment of administration or immediately prior thereto. The preparation can be made, for example, by means of a syringe or a pump system which enables the two aforesaid compounds to be mixed together at the moment of administration or immediately prior thereto. The administration can be carried out, for example, by means of a dual syringe and/or cosmetic pump, e.g., by means of a pump marketed by the Kemai Company (trademark).

In other words, even though in this description reference is made to a composition, it is well understood that each of the compounds of the composition can be administered jointly or concomitantly with the other compounds (e.g., in a single composition or in two compositions or in three compositions, each of these compositions including one or more of the aforesaid components, the method of administering each of the compounds or composition being optionally identical or different), or independently of one another, e.g., successively, e.g., separate administration of at least one inhibitor of hydroxymethylglutaryl-coenzyme A (HMG-CoA) reductase, separate administration of at least one inhibitor of farnesyl pyrophosphate synthase, these administrations being carried out with respect to a single patient, jointly, concomitantly or successively or alternately, in the aforesaid order or in another order. These various administrations can be carried out independently of one another or in an interrelated manner (composition or co-administration), by an identical or different method of administration (injection, ingestion, topical application, etc.), one or more times per day, for one or more successive or non-successive days.

This composition, for example, can be intended for the treatment of pathological or non-pathological situations associated with the accumulation and/or persistence of prenylated proteins in cells.

It is also within the scope of the invention to use compounds which are both inhibitors of hydroxymethylglutaryl-coenzyme A (HMG-CoA) reductase and inhibitors of farnesyl pyrophosphate synthase.

Very particularly, the cosmetic and/or dermatological composition according to the invention can be intended for the treatment of pathological or non-pathological situations associated with the accumulation and/or persistence of progerin in cells; even more particularly, for the treatment of situations associated with the accumulation and/or persistence of farnesylated prelamin A in cells, whether truncated or non-truncated or modified.

In particular, since it is admitted that physiological ageing is in particular a consequence of the presence of progerin in cells over the course of life. The progerin is concentrated in particular in the mesenchymal cells, the composition according to the invention can be intended to prevent the effects of cell ageing, particularly in the skin and/or vascular endothelium.

For example, said cosmetic composition can be used in order to prevent and/or treat skin disorders. These skin disorders can be disorders of natural or pathological origin or associated with a therapeutic treatment. The disorders of natural origin are those associated with natural ageing, e.g., skin ageing. The pathological disorders, for example, are those associated with hormonal skin ageing, photo-induced ageing, premature skin ageing, myolipocutaneous ageing, lipodystrophy and restrictive dermopathy. The disorders associated with a treatment, for example, are those associated with chemotherapeutic treatments, X-ray irradiation by X-rays, gamma rays, ultraviolet rays or those associated with side effects of the treatments.

Progeria is accompanied by very pronounced alopecia. Afflicted children lose their hair starting at 2 years old, which hair becomes sparse, white and silky. The eyelashes and eyebrows also fall out, and body hair is rare or completely absent (see the detailed description of clinical symptoms compiled by R. Hennekam (2006). This alopecia is likewise observed in the murine model of progeria (Zmpste24-deficient mice). In a completely unexpected way, the treatment with an association of pravastatin and zoledronate according to this invention at least partially restores the fur coat of these mice.

The cosmetic and/or dermatological composition of this invention can be intended for the treatment of any living being, man or animal, particularly for preventing the effects of cell ageing. The composition therefore finds application in both human medicine and veterinary medicine, in cosmetics and/or in dermatology. For example, it makes it possible to repair and/or prevent physiological or non-physiological skin disorders, including ageing of the skin and the alteration or disappearance of the hair.

According to the invention, any inhibitor of farnesyl pyrophosphate synthase, or of one of the physiologically acceptable salts thereof, can be used in the preparation of the composition according to the invention.

The physiologically acceptable salts, for example, can be salts formed with hydrochloric, hydrobromic, nitric, sulphuric, phosphoric acids, carboxylic acids, e.g., such as acetic, formic, propionic, benzoic, maleic, fumaric, succinic, tartaric, citric, oxalic, glyoxylic and aspartic acids, sulfonic alkanes such as sulfonic methane or ethane acids, arylsulfonic acids such as benzene or para toluene sulfonic acids.

In particular, the inhibitor of farnesyl pyrophosphate synthase can be one of the members of the family of polyphosphonates, particularly aminobiphosphonates (NBPs), or one of the physiologically acceptable salts thereof.

Polyphosphonates are synthetic molecules widely used in the treatment of osteoporosis and for bone regeneration.

The term phosphonate applies to molecules very similar to phosphate:

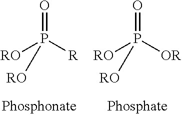

The core structure of biphosphonates (BPs) is the equivalent of a P—O—P bond in ATP, for example, but where the oxygen is replaced by a carbon. This endows these molecules with a completely distinctive degree of stability.

A simple biphosphonate would be equivalent to ADP, the two phosphate groups (O₃P—) being replaced by the biphosphonate group.

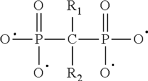

Unlike the oxygen of the phosphates, the central carbon can also be involved in 2 bonds, and it is the nature of the groups grafted onto this carbon which determines the specificity of the biphosphonates.

When the “side” chains (R1 and R2) comprise an amine function (NH), or more generally one or more nitrogen atoms, reference is made to an aminobiphosphonate or NBP.

Of course, other substituents can be attached to the oxygen atoms.

Pyrophosphoric acid or pyrophosphate in solution (PPi)

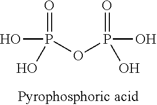

is used in numerous metabolic reactions as a substrate transporter, and it is restored at the end of the reaction. One of the metabolic pathways using molecules coupled with pyrophosphate is that of protein prenylation.

Grafting of an isopentenyl-PP (base unit at C5) onto a geranyl-PP (C10) in order to yield a farnesyl-PP, which is a reaction catalyzed by the farnesyl pyrophosphate synthase (FPS) enzyme, releases a PPi.

It is this step which is specifically inhibited by the NBPs.

In this regard, and as an example, the aminobiphosphonate (inhibitor of the farnesyl pyrophosphate synthase) can be chosen from amongst:
alendronic acid or the ionic form thereof, alendronate;
clodronic acid or the ionic form thereof, clodronate;
etidronic acid or the ionic form thereof, etidronate;
ibandronic acid or the ionic form thereof, ibandronate;
medronic acid or the ionic form thereof, medronate;
neridronic acid or the ionic form thereof, neridronate;
olpadronic acid or the ionic form thereof, olpadronate;
pamidronic acid or the ionic form thereof, pamidronate;
risedronic acid or the ionic form thereof, risedronate;
tiludronic acid or the ionic form thereof, tiludronate;
zoledronic acid or the ionic form thereof, zoledronate;
4-N,N-dimethylaminomethane diphosphonic acid or the ionic form thereof, dimethylaminomethanediphosphonate;
α-amino-(4-hydroxybenzylidene)diphosphonate.

Preferentially, according to the invention, it is preferred to use zoledronic acid (also called zolendronic acid) or the ionic form thereof, zoledronate (also called zolendronate).

According to the invention, any HMG-CoA reductase inhibitor, or one of the physiologically acceptable salts thereof, can be used in preparing the compositions.

In particular, the HMG-CoA reductase inhibitor can be a molecule of the family of statins, whether liposoluble or water-soluble, or one of the physiologically acceptable salts thereof.

Statins have been shown to exist in fungi. They have an inhibiting activity with respect to the HMG-CoA reductase, a key enzyme in the biosynthesis of cholesterol and steroids, which catalyzes the reduction of the hydroxymethylglutarate coupled with the Coenzyme A into mevalonic acid (mevalonate in solution). This inhibition is ensured by their structural analogy with the skeleton of hydroxymethylglutarate. The metabolic pathway involved is admittedly that of cholesterol biosynthesis, but is also that of the synthesis of phenyl groups, which are 5-carbon isoprene base unit polymers used to modify approximately 300 proteins in cells and to attach a lipophilic tail thereto, enabling, in particular, the anchoring of same to the membranes.

The principle polyprenes, all of which are derived from pyruvate and HMG-CoA, are geranyl (C10), farnesyl (C15) and geranylgeranyl (C20).

All of the statins are globally hepatoselective, but all do not have the same mode of entry into cells. As a matter of fact, pravastatin and rosuvastatin are both hydrophilic, and therefore water-soluble, on the other hand, all of the others are lipophilic and therefore can freely diffuse through the plasmic membranes (double layers of lipid molecules), which undoubtedly explains the higher degree of toxicity thereof. Water-soluble statins need a specific transporter in order to enter the cell, Organic Anion Transporter 3, or OAT3, or SLC22A8 (Takedaa et al., 2004 (46)).

They are widely used for treating hypercholesterolemia, and the side effects thereof, which are rare, are well characterized. This is the case in particular of rhabdomyolysis (1 to 7% of the cases depending on the molecule used, Evans et al., 2002 (12)), the early signs of which are muscle pain in the patient being treated and result in the immediate cessation of treatment.

In this regard, and as an example, a statin can be chosen from amongst atorvastatin, simvastatin, pravastatin, rivastatin, mevastatin (or compactin), fluindostatin, velostatin, fluvastatin, dalvastatin, cerivastatin, pentostatin, rosuvastatin, lovastatin, pitavastatin, or one of the physiologically acceptable salts thereof.

Lovastatin, pravastatin and simvastatin are molecules derived from fungal metabolites, whereas the others (atorvastatin, cerivastatin, fluvastatin, pitavastatin and rosuvastatin) are entirely synthetic. According to the invention, pravastatin is preferably used, which is a water-soluble semi-natural statin.

According to the invention, it is of course possible to use one, two or more inhibitor(s) of farnesylpyrophosphate synthase associated with one, two or more inhibitor(s) of HMG-CoA reductase.

According to one particular form of the invention, the composition can be intended for the treatment of pathological or non-pathological situations requiring protein prenylation to be inhibited. These diseases may or may not be labelled, e.g., Noonan's syndrome, cardio-fascio-cutaneous syndrome, or diseases associated with abnormal prenylation or persistence of Ras and signal transduction proteins.

According to another particular form of the invention, the composition can be intended for the treatment of pathological or non-pathological situations exhibiting signs of ageing, whether natural, premature or accelerated. In particular, in cases of signs of deterioration of the vascular endothelium (protection of the vascular endothelium), ageing of the skin, hair loss of body hair and/or head hair, and bone lysis.

The composition according to the invention is preferably a dermatological and/or cosmetic composition intended for the treatment of Progeria (HGPS, Hutchinson-Gilford Progeria Syndrome) and restrictive dermopathy (DR or RD).

According to the invention, the inhibitor of farnesylpyrophosphate synthase and the inhibitor of HMG-CoA reductase are advantageously present in the composition at physiologically effective doses.

Generally speaking, the quantities to be administered can be adapted based on the patient, the disease, the method of administration, etc. It is understood that repeated applications can be carried out, possibly in combination with other active ingredients or any vehicle.

In general, the daily dose of the inhibitors will be the maximum dose for obtaining the desired dermatological and/or cosmetic effect.

According to the invention, the inhibitor of hydroxymethylglutaryl-coenzyme A (HMG-CoA) reductase and the inhibitor of farnesylpyrophosphate synthase can be used in the composition, mixed with one or more inert excipients or vehicles, i.e., physiologically inactive and nontoxic. Reference can be made, for example, to the ingredients commonly used in cosmetic or dermatological preparations.

The cosmetic and/or dermatological composition of this invention includes at least one cosmetic and/or dermatological product. A “cosmetic and/or dermatological product” is understood to mean any product which makes it possible to form a composition, together with the aforesaid inhibitors, enabling a cosmetic and/or dermatological application, i.e., a composition capable of being applied to the skin or enabling treatment of the skin from a cosmetic and/or dermatological viewpoint.

At a minimum, this cosmetic and/or dermatological product is that enabling the two inhibitors to be suspended or solubilised so as to be able to apply same to the skin. For example, in this case, it may involve an alcohol, water, a buffer, a saline solution, an isotonic solution, or a mixture of two or more of same.

Preferably, of course, the cosmetic and/or dermatological product is a physiologically acceptable product, for topical use in particular.

The cosmetic and/or dermatological product usable in the composition of this invention can likewise include or consist of any compound or mixture of compounds routinely used in cosmetic and/or dermatological compositions.

For example, this can involve one or more compounds chosen from the group including surfactants, thickening agents, gelling agents, preservatives, humectants, emulsifiers, perfume, silicones, chelating agents, antioxidants, cosmetic dyes, fungicides, antibacterial agents, film-forming agents, stabilizers, buffering agents, UV filters, binders and emulsion stabilizers.

An “emulsifier” is understood to mean any compound enabling mixtures of non-miscible liquids to be promoted. These compounds are known to a person skilled in the art. For example, the emulsifier can be chosen from the group including cationic and anionic substances, e.g., fatty acids and glycols, e.g., butylene glycol, propylene glycol, polyethylene glycol, polyglucosides, plant sterols, stearic alcohols, behenet-25 and cetearyl alcohol.

A “preservative” is understood to mean any compound enabling the development of microorganisms in a cosmetic and/or dermatological composition to be inhibited. These compounds are known to person skilled in the art. For example, this may involve a fungicide and/or an antibacterial agent.

A “fungicide” is understood to mean any compound capable of reducing and/or inhibiting the proliferation of fungi in a cosmetic and/or dermatological composition.

An “antibacterial agent” is understood to mean any compound capable of reducing or inhibiting the proliferation of bacteria in a cosmetic and/or dermatological composition. For example, the preservative can be chosen from the group including parabens, e.g., butylparaben, ethylparaben, propylparaben, isobutylparaben, methylparaben, organic acids, diazolidiines, isothiazolinones, hydroxymethylglycinates, phenol alcohols, 5-bromo-5-nitro-1,3, dioxane, diazolidinyl urea, 3′-Demethoxy-3O-Demthylmatairesinol (DMDM) hydantoin.

For example, the silicone can be chosen from the group including dimethicones, dimeticolols, phenyl trimethicones and volatile and non-volatile silicone oils.

A “chelating agent” is understood to mean any compound forming complexes with metal ions modifying the stability and/or viscosity of a cosmetic and/or dermatological composition. These compounds are known to a person skilled in the art. For example, the chelating agent can be chosen from the group including nitroloacetic acid, polyaminocarboxylic acid, ethylenediamine tetraacetic acid (EDTA) and salts thereof.

A “thickening agent” is understood to mean any compound capable of increasing the viscosity of a cosmetic and/or dermatological composition. These compounds are known to a person skilled in the art. For example, the thickening agent can be chosen from the group including natural polymers like alginic acid and the derivatives thereof, cellulose and the derivatives thereof, scleroglucans, xanthan gum, synthetic carboxyvinyl polymers and copolymers which may likewise have an emulsifying function, e.g., acrylate, methacrylate, acrylamide, and the mixtures thereof, e.g., the alkyl acrylate/acrylate copolymer at C10 to 30, and polymethlymethacrylate.

A “humectant” is understood to mean any compound capable of maintaining and/or preserving the moisture of a cosmetic and/or dermatological composition. These compounds are known to a person skilled in the art. For example, the humectant can be chosen from amongst butylene glycol, propylene glycol and polyethylene glycol.

An “antioxidant” is understood to mean any compound capable of inhibiting oxygen oxidation reactions and rancidity. These compounds are known to a person skilled in the art. For example, the antioxidant can be chosen from the group including sulphites, amino acids, e.g., glycine, histidine, tyrosine, tryptophan and derivatives thereof, imidazols, peptides, e.g., D-carnosine and L-carnosine.

A “surfactant” is understood to mean any compound making it possible to reduce the surface tension of the composition and to promote the uniform distribution of the compounds of a cosmetic and/or dermatological composition. These compounds are known to a person skilled in the art. For example, the surfactant can be chosen from the group including cetyl betaine, cocomidopropyl betaine, disodium cocoamphodiacetate, disodium laureth sulfosuccinate, hexyleneglycol, laureth-3, laureth-4, polyethylene glycol (PEG) PEG-30, glyceryl cocoate, PEG-40 hydrogenated castor oil, PEG-7, glyceryl cocoate, polysorbate 20, polysorbate 80, potassium cetyl phosphate, sodium cetearyl sulfate, sodium chemical laureth sulfate, sodium lauryl sulfate, sodium palmitate (sodium hexadecanoate) steareth-100, steareth-2 and steareth-21.

An “absorbent” is understood to mean a compound which absorbs dissolved or dispersed water-soluble and/or liposoluble substances. These compounds are known to a person skilled in the art. For example, the absorbent can be chosen from the group including boron nitride, silicic acid, magnesium carbonate, microcrystalline cellulose, silica (silicon dioxide) and talc (E553).

A “film-forming agent” is understood to mean a compound which enables the formation of a continuous film during application of a cosmetic and/or dermatological composition. These compounds are known to a person skilled in the art. For example, the film-forming agent can be chosen from the group including acrylamide/ammonium acrylate copolymer, acrylamide copolymer, acrylamidopropyltrimonium/acrylate chloride copolymer, acrylateacrylate/alkyl C10-30 acrylate copolymer, white beeswax (E901), cyamopsis tetragonolobus (Guar), hydroxymethylcellulose, polyethylene terephthalate (PET), polygylcerylmethacrylate, polyquaternium-10, polyquaternium-11, polyquaternium-7, polyvinylpyrrolidone (PVP)/Eicosene copolymer, PVP/Hexadecene copolymer, PVP/vinylacetate (VA) copolymer.

A “stabilizing agent” is understood to mean a compound which enables the stability of a cosmetic and/or dermatological composition to be improved and/or enables the stability of the components included in said composition to be improved. These compounds are known to a person skilled in the art. For example, the stabilizing agent can be chosen from the group including hydroxyethylcellulose, microcrystalline cellulose, polycaprolactone and sodium stannate.

A “buffering agent” is understood to mean a compound which enables the pH of a cosmetic and/or dermatological composition to be improved. These compounds are known to a person skilled in the art. For example, the buffering agent can be chosen from the group including aminomethyl propanol, citric acid, fumaric acid, orthophosphoric acid, sebacic acid (decanedioic acid), sodium citrate, sodium hydroxide, tartaric acid, tetrasodium pyrophosphate and triethylamine (TEA).

A “UV filter” is understood to mean a compound enabling certain UV rays to be filtered. These compounds are known to a person skilled in the art. For example, the UV filter can be chosen from the group including benzophenone-3 (oxybenzone), benzophenone-4 (sulisobenzone), avobenzone, ethylhexylmethoxycinnamate, octylmethoxycinnamate and ethylhexyl salicylate.

A “moisturizer” is understood to mean an compound enabling the water content of a cosmetic and/or dermatological composition to be increased. These compounds are known to a person skilled in the art. For example, the moisturizer can be chosen from the group including aloes (aloe vera), pineapple plant extract, dimethiconol, Echinacea angustifolia, glycine, liquorice extract, jasmine extract, jojoba esters, magnesium stearate, mannitol, honey and castor oil.

A “binder” is understood to mean a compound enabling the cohesion of a cosmetic composition to be increased. These compounds are known to a person skilled in the art. For example, the binder can be chosen from the group including acrylate copolymers, cyamopsis tetragonolobus (Guar), hydroxymethylcellulose, isopropyl myristate, magnesium carbonate, mannitol, polyethylene glycol-90M, polydecene, PVP/Eicosene copolymer, PVP/Hexadecene copolymer, PVP/VA copolymer, sodium acrylate copolymer, sodium carboxymethyl betaglucan copolymer, sodium carboxymethyl dextran, paraffin wax and xanthan gum.

An “emulsion stabilizer” is understood to mean a compound making it possible to improve the emulsification of a cosmetic and/or dermatological compound and/or to improve the stability of a cosmetic and/or dermatological emulsion. These compounds are known to a person skilled in the art. For example, the emulsion stabilizer can be chosen from the group including acetylated glycol stearate, carbomer, alcohol cetearyl, cyamopsis tetragonolobus, hydroxyethylcellulose, microcrystalline cellulose, PEG-90M, PVP/VA copolymer, sodium carboxymethyl dextran, stearic acid, octadecanol, synthetic paraffin wax and xanthan gum.

A “perfume” is understood to mean a compound making it possible to give a cosmetic and/or dermatological composition a particular odour. These compounds are known to a person skilled in the art. For example, the perfume can be any appropriate perfume for cosmetic and/dermatological use. The perfume can be chosen, for example, from the group including natural plant extracts, plant essences, e.g., extracts and/or essences of roses, chamomile and mint.

The compound(s) used to make the cosmetic and/or dermatological product depend, in particular, on the formulation chosen for the cosmetic and/or dermatological use of the composition of this invention.

The cosmetic and/or dermatological composition can be in any form appropriate for the use thereof. For example, it may involve a solution, a suspension, an emulsion, a microemulsion or a paste. For example, it may involve a gel, a cream, an aerosol, an ointment, a foam, a powder, an oil, tablets, suppositories, gel capsules, capsules, etc., possibly in galenic form, or a device ensuring extended and/or delayed release. For this type of formulation, an agent such as cellulose, carbonates or starch is advantageously used.

The cosmetic and/or dermatological composition of the invention can likewise include water. In this case, the composition preferably includes demineralised or distilled water.

Said composition can likewise include at least one other active ingredient, particularly another therapeutically and/or cosmetically and/or dermatologically active ingredient, e.g., for use simultaneously, separately or spread out over time, depending on the galenic formulation used.

The composition can further include a means enabling the penetration thereof into the skin to be facilitated. For example, a compound chosen from the group including vasodilator and glucocorticoids. This is beneficial when the composition of this invention is intended for topical use.

The composition of this invention can be used via any appropriate means known to a person skilled in the art. For example, for topical, transdermal or transcutaneous use, or for a composition administered orally. It can likewise be used in a transdermal patch. It can likewise be applied to the skin by means of a sprayer or by dipping/soaking.

To apply the composition, a physical means can also be used, which increases the penetration of the composition into the skin, e.g., a plastic film deposited on or enveloping the skin after application of the composition.

According to the invention, in the cosmetic and/or dermatological composition of the invention, the ratio of farnesylpyrophosphate synthase inhibitor to hydroxymethylgultaryl coenzyme A reductase inhibitor, or one of the physiologically acceptable salts thereof, can be between 0.002 and 50, preferably between 0.005 and 25, preferably between 0.01 and 1.8, and preferably from 0.05 to 0.35.

In the cosmetic and/or dermatological composition of the invention, the quantity of farnesylpyrophosphate synthase inhibitor can between 0.001 to 0.500 percent by weight, preferably from 0.005 to 0.025, preferably from 0.025 to 0.1, preferably from 0.100 to 0.150, preferably from 0.150 to 0.200, preferably from 0.200 to 0.250, preferably from 0.250 to 0.300, preferably from 0.300 to 0.350, preferably from 0.350 to 0.400, preferably from 0.400 to 0.450, and preferably from 0.450 to 0.500 percent by weight of the cosmetic and/or dermatological composition.

In the cosmetic and/or dermatological composition of the invention, the quantity of hydroxymethylgultaryl coenzyme A reductase inhibitors can be between 0.010 to 0.500 percent by weight, preferably from 0.075 to 0.095, preferably from 0.095 to 0.100, preferably from 0.100 to 0.150, preferably from 0.150 to 0.200, preferably from 0.200 to 0.250, preferably from 0.250 to 0.300, preferably from 0.300 to 0.350, preferably from 0.350 to 0.400, preferably from 0.400 to 0.450, and preferably from 0.450 to 0.500 percent by weight of the cosmetic and/or dermatological composition.

In the cosmetic and/or dermatological composition of the invention, the sum of the quantity of hydroxymethylgultaryl coenzyme A reductase inhibitors and farnesylpyrophosphate synthase inhibitor can be between 0.010 and 1.000, preferably from 0.1 to 0.800 percent by weight of the composition, and preferably the quantity is equal to 0.1 percent by weight.

Other advantages may become further apparent to a person skilled in the art upon reading the following examples, given for illustrative purposes and shown in the appended figures.

### BRIEF DESCRIPTION OF THE FIGURES

FIG. 1 shows the Western Blot results obtained from “normal” control fibroblasts treated with increasing doses of a water-soluble statin (pravastatin P, 20 to 100 μM), and of an aminobiphosphonate (NBP, zoledronate Z, 20 to 100 μM) (Tracks A to I, P20/Z20, P20/Z60, P20/Z100, P60/Z20, P60/Z60, P60/Z100, P100/Z20, P100/Z60 AND P100/Z100, respectively). Track J is a positive control for the presence of prelamin A (fibroblasts of RD patients), track K is the negative control, treated with the solvent alone (PBS).

FIG. 2 shows the results obtained at the effective doses for each of the products.

FIG. 3 shows the superior effect obtained when administering the 2 products together.

FIG. 4 shows the action of the association of the 2 products on aged cells.

FIG. 5 shows the cell multiplication of fibroblasts by measuring the mitotic index in relation to the cell culture conditions. The mitotic index corresponds to the ratio between the number of tagged nuclei (being divided) relative to the total number of nuclei in the field observed with respect to each treatment.

FIG. 6: Western blot showing that blocking the prenylation of prelamin A requires the inhibition of both the farnesyltransferase and the type I genaraylgeranyltransferase. Detection of the lamin A/C in HeLa cells treated with the farnesyltransferase and/or type I geranylgeranyltransferase. LA=lamin A, LC=lamin C, Pre=prelamin A.

FIG. 7: Lamin A (control cells) and prelamin A (Zmpste24^{-/-} mouse cells) were analyzed by mass spectrometry (MALDI-ToF). a, b: portions of the spectrum corresponding to the farnesylated (a) and geranylgeranylated (b) peptides.

FIG. 8: Mass spectrometry analysis of proteins extracted from the nuclear envelope of untreated cells 9a), of progeria patient cells treated with FTI (2.5 μM each, c).

FIG. 9: Mass spectrometry analysis of proteins extracted from the nuclear envelope of untreated fibroblasts (a) or ones treated with the pravastatin+zoledronate mixture (1 μM each, b) derived from Zmpste24^{-/-} mice.

FIG. 10: illustrations of various experiments showing the synergistic effect of the pravastatin+zoledronate combination on the accumulation of prelamin A in control cells and in those of progeria patients: 9a) Immunocytochemical detection of lamin A/C and of prelamin A in normal human fibroblasts untreated or treated with pravastatin and/or zoledronate. (b) Immunocytochemical detection of lamin A/C and prelamin A in normal human fibroblasts and those of progeria patients treated with the pravastatin+zoledronate combination. (c) Quantitative analysis of the effect of the pravastatin+zoledronate treatment on the nuclear morphology of progeria patient cells. (d) Quantitative analysis of the effect of the pravastatin+zoledronate treatment on the nuclear morphology of progeria patient cells in the presence of farnesol, geranylgeraniol or of both compounds. Error bars=mean±standard error of the mean. Scale bar=10 μm.

FIG. 11: Illustration of the pravastatin+zoledronate treatment results showing correction of the nuclear morphology and induction of a partial relocalization of the isoforms of lamin A/C and of lamin B1 of the nuclear lamina in the nucleoplasm, in progeria patient cells. (A) Immunofluorescence and confocal microscopy. Images a to c of each panel are projections of the average intensity of 27 images of the stack and show the tubules of the nuclear reticulum tagged by the calreticulin in the progeria cells incubated with PBS. Images d to I: isolated confocal sections of 0.2 μm in thickness. Effect of the pravastatin+zoledronate treatment (g) and (h). Colocalization of lamin B1 and the calrecticulin. Scale bar=5 μm.

FIG. 12: Illustration of the effect of pravastatin and zoledronate combined or uncombined on the nuclear morphology of Zmpste24^{-/-} mice cells 9a) and of control mice (b) in culture, in the presence of farnesol, geranylgeraniol and of both molecules.

FIG. 13: Effect of the pravastatin and zoledronate treatment on the double-stranded break (DSB) anomalies of the DNA in the cells of progeria patients. Immunodetection of the foci of the phosphorylated histone H2AX detected 24 hours after irradiation, foci corresponding to the unrepaired double-stranded breaks (top images). Nuclear tagging of the DAPI (bottom images). Bottom curves: evolution of the number of foci of phosphorylated histone H2AX in relation to the time after irradiation in the control cells (solid square) and the progeria cells (empty square) incubated with PBS or treated with pravastatin+zoledronate. Each curve represents the mean±standard error of the mean at least 3 experiments.

FIG. 14: Effect of the pravastatin+zoledronate treatment on the progeroid phenotype of Zmpste24^{-/-} mice: (a) Photographs representative of 3-month old Zmpste24^{+/+}, Zmpste24^{-/-} mice and Zmpste24^{-/-} mice treated with the pravastatin+zoledronate combination. Scale bar=1 cm. (b) Weight of 3-month old Zmpste24^{+/+} mice (n=12), Zmpste24^{-/-} (n=13) and treated Zmpste24^{-/-} (n=15). (c) Kaplan-Meier curves showing a significant increase in the lifespan of the treated Zmpste24^{-/-} mice. (d) Three-dimensional computed microtomography illustration of the tibia of treated and untreated Zmpste24^{-/-} mice (top image). The bottom panel shows the relative bone volume and the number of trabecular bones in the treated and untreated Zmpste24^{-/-} mice. (e) Quantification of the nuclear anomalies of the hepatocytes of Zmpste24^{+/+} Zmpste24^{-/-} mice and treated Zmpste24^{-/-} mice. The white arrows show the abnormal nuclei. Scale bar=10 μm. (f) Relative expression of the p53 target genes in the liver and heart of Zmpste24^{+/+} Zmpste24^{-/-} mice and treated Zmpste24^{-/-} mice, analyzed by quantitative RT-PCR. * P<0.05; ** P<0.01; *** P<0.0001. The error bars represent the mean±standard error of the mean.

FIG. 15: Effect of pravastatin alone or of zoledronate on the lifespan of Zmpste24^{-/-} mice; Kaplan-Meier curves with pravastatin alone (a) and zoledronate alone (b) on treated (empty diamond) and untreated (solid circles) Zmpste24^{-/-} mice.

FIG. 16: Effect of a pravastatin+zoledronate treatment on the lifespan of Linna^{-/-} mice: Kaplan-Meier curves of Linna^{-/-} mice treated with pravastatin+zoledronate (n=12, empty diamond), compared with that of untreated mice (solid circles, n=11).

### EXAMPLES

### Example 1

Synergistic effect of the association of an inhibitor of hydroxymethylglutaryl-coenzyme A (HMG-CoA) reductase (a water-soluble statin: pravastatin) and an inhibitor of farnesylpyrophosphate synthase (an aminobiphosphonate: zoledronate) on normal and progeroid cell cultures.

### Protocols

### Cells and Cell Culture

The cell lines are either control fibroblasts AG16409 derived from the Coriell Institute, or fibroblasts derived from biopsies of patients afflicted with Restrictive Dermopathy. They are cultivated at 37° C. under 5% CO₂ in clean room P2.

The usual complete culture medium is:
RPMI (Invitrogen) supplemented with
20% Foetal Calf Serum (Invitrogen)
L-Glutamine 200 mM (Invitrogen)
Penicillin/Streptomycin/Fungizone Mixture 1× (Stock 100×, Cambrex).

### Harvesting of the Cells

The cells are harvested by trypsinization in the following way (protocol for a large flask, 75 cm², BD Falcon):
The medium is aspirated;
The cells are washed with 10 ml of 1× PBS (Invitrogen);
5 ml of a solution of 1× Trypsin/EDTA (Cambrex) is added;
The flask is incubated for approximately 6 min at 37° C., the time required for the cells to break loose;
The trypsin is inhibited by dilution in 15 ml of complete medium;
The cells are formed into a pellet via centrifugation for 10 min at 1,000 revolutions per minute at 16° C.;
The pellet is resuspended in 2 ml of 1× PBS, and re-centrifuged under the same conditions.

The cells obtained are either frozen for later use or subcultured from this washed pellet.

### Treatments

The pravastatin solution (water-soluble statin) used is prepared as follows:

40 mg of pravastatin (Sigma Aldrich, P4498) are taken up in sterile water to form a 10 mM stock solution.

The final tested concentrations were 500 nM, 1, 5, 50 and 100 μM, which were obtained by diluting the stock solution in the complete medium.

The zoledronate solution (NBP) used is prepared as follows:

A stock solution of (1-hydroxy-2-imidazol-1-yl-1-phosphono-ethyl)phosphonic acid (0.8 mg/ml, Novartis) is adjusted to a concentration of 2 mM.

The final tested concentrations were 500 nM, 1, 5, 50 and 100 μM, which were obtained by diluting the stock solution in the complete medium.

### Western Blot

### Preparation of the Cells

For a Western Blot experiment, the cells are treated as follows:

Approximately 7.5×10⁵ cells are seeded in a large flask and cultivated under the above conditions until near confluency (4 days).

At the end of 4 days, the cells are washed with 1× PBS, and taken up in a complete medium supplemented with the treatment.

The cells are incubated for the treatment time (from 6 to 72 hours, sequentially or simultaneously) in the incubator at 37° C.

At the end of the treatment, the cells are trypsinized (above protocol) and the resulting pellet is stored at −80° C. until the proteins are extracted.

### Extraction of the Proteins for Western Blot

The cell pellet is taken up in 300 μl of a lysis buffer:

| | |
|---|---|
| Triton X 100 | 1% |
| SDS | 0.1% |
| Sodium desoxycholate | 0.5% |
| NaCl | 50 mM |
| EDTA | 1 mM |
| TrisHCl pH 7.4 | 20 mM |
| Protease inhibitor | 1 tablet per 50 ml |
| (Roche 11697498001) | |
| Extemporaneous addition of | 1 mM |
| Sodium orthovanadate | |
| PMSF | 1 mM |

The cells are exposed to sonication twice for 30 sec (Brandson Sonifier Cell Disruptor B15).

The cell debris is centrifuged for 10 min at 10,000 revolutions per minute at 4° C.

The protein supernatant is preserved at −80° C. until used.

Protein analysis is carried out upon thawing.

### Western Blot

### Gel

A gel of 8% acrylamide is conventionally used for detecting the various forms of lamins A/C.

| | |
|---|---|
| Acrylamide/bisacrylamide 37/1 | 8% |
| TrisHCl pH 8.8 | 375 mM |
| SDS | 0.1% |
| APS | 0.1% |
| TEMED | 0.01% |

A stacking gel is poured over the resolving gel

| | |
|---|---|
| Acrylamide/bisacrylamide 37.5/1 | 3% |
| TrisHCl pH 6.8 | 375 mM |
| SDS | 0.1% |
| APS | 0.1% |
| TEMED | 0.01% |

The protein concentration of the samples is determined, and aliquots are adjusted to 50 μg per tube in a lysis buffer in QS 15 μl.

5 μl of a loading buffer are added to each sample:

| | |
|---|---|
| SDS | 4% |
| TrisHCl pH 6.8 | 100 mM |
| Glycerol | 20% |
| β-mercaptoethanol | 20% |
| Bromophenol blue | trace amounts |

The samples are denatured by heating for 5 min at 95° C. and deposited in the wells.

The migration occurs at 50 and then 100 Volts, in a buffer:

| | |
|---|---|
| Tris-Base | 0.3% |
| Glycine | 1.44% |
| SDS | 0.1% |

### Transfer

The transfer membrane (Hybon P, Amersham Biosciences) is prepared by soaking in ethanol, followed by a bath in sterile water and for 10 min in the transfer buffer:

| Tris-Base | 12 mM |
|---|---|
| Glycine | 96 mM |
| Ethanol | 20% |

The gel is moistened for 20 min in the transfer buffer, and then the sandwich is assembled (Miniprotéan system, Biorad).

The transfer generally occurs at night, in a cold chamber at 10 Volts.

The membrane is rinsed in 1× PBS, kept moist, and used as is for the detection operation.

### Detection

The membrane is incubated for 1 hour at ambient temperature in a saturating solution:

| | |
|---|---|
| Casein | 10% |
| Tween 20 | 0.1% |
| PBS | 1 X |

It is rinsed twice for 10 min in a washing buffer (Tween 20 0.1%/1× PBS).

The primary antibody is diluted in the saturating buffer (for details and dilution, see immunolabelling below).

The membrane is incubated with the primary antibodies for 1 hour at ambient temperature under agitation.

Upon completion of this, it is rinsed 3× with a washing buffer, then washed 3× for 15 min with the same buffer.

The secondary antibody (peroxidase-coupled system, Jackson Immunoresearch) is diluted to 1/10,000^{th} in a saturating buffer.

The membrane is incubated with this solution for 30 to 45 min at ambient temperature under agitation.

Upon completion of this, it is rinsed 3× with a washing buffer, then washed 3× for 15 min with the same buffer.

The detection operation is carried out with the ECL Plus Western Blotting System kit from Amersham Bioscience, according to the supplier's instructions.

After developing, the membrane is exposed on Biomax MR (Kodak) film, the time required in order to obtain a satisfactory signal.

### Immunolabelling

### Cell Preparation

A cell culture is trypsinized and the cells counted in a Neubauer chamber.

Labtech style culture wells (Nunc, Ref. 177399) are seeded, at the rate of 5×10⁴ cells per well.

The complete culture medium is supplemented with the treatment or treatments (statin, NBP, both), and the cells are cultivated for the ad hoc time.

Upon completion of this, the culture medium is aspirated and the wells disassembled.

The slides are washed in 1× PBS.

The cells are fixed in a 4% paraformaldehyde solution (in PBS) for 10 minutes at ambient temperature.

A 10-min wash in 1× PBS is carried out.

The cells are dehydrated by successive 3-min baths in solutions containing an increasing percentage of ethanol (70, 90, 100%, the last bath being repeated).

After drying, the slides are stored at −80° C. until used.

### Labelling

After thawing, the cells are incubated for 5 min at ambient temperature in a moisture chamber in 50 μl of a permeabilization solution:

| | |
|---|---|
| PBS | 1 X |
| Triton X 100 | 0.5% |
| RNS | 5% |
| (Normal Rabbit Serum, Vector S5000) | |
| Protease inhibitor | 1 tablet per 50 ml |
| (Roche 11697498001) | |

3 preincubation baths of 15 min each are carried out in 50 μl of the incubating solution:

| | |
|---|---|
| PBS | 1 X |
| RNS | 5% |
| Protease Inhibitor | 1 tablet per 50 ml |
| (Roche 11697498001) | |

The primary antibody is diluted to 1/100^{th} in 50 μl of incubating solution and placed in contact with the cells for 1 hour at ambient temperature in a moisture chamber.

The primary antibodies used are of 2 types:
Anti-lamin A/C mouse (N-terminal side), clone 4A7, donation from G. Morris (Oswestry, UK)
Anti-prelamin A goat (15 aa C-terminal end), product SC6214, Santa Cruz Biotechnology, Inc.
3 rinsings of 15 min each are carried out in 50 μl of 1× PBS.

The incubation with the secondary antibody occurs for 1 hour in 50 ml of incubating solution at ambient temperature in a moisture chamber. The secondary antibodies are of two types:
Donkey anti-mouse, Jackson Immunoresearch, dilution to 1/100^{th}
Donkey anti-goat, Jackson Immunoresearch, dilution to 1/200^{th}.

3 rinsings of 15 min each are carried out in 50 μl of 1× PBS.

Incubation with 100 μl of 50 ng/ml DAPI solution (SERVA, Ref. 18860) is carried out for 15 min at ambient temperature in a moisture chamber.

3 rinsings of 5 min each are carried out in 1× PBS in slide-holder trays.

A final rinsing is carried out for 5 min in a 0.1% Tween20 solution in PBS.

### Mounting

The cells are immersed in a drop of VectaShield (Vector), covered with a cover glass and observed under a fluorescence microscope (Leica DMR, Leica Microsystems), equipped with a coolSNAP camera system (Princeton).

### Results

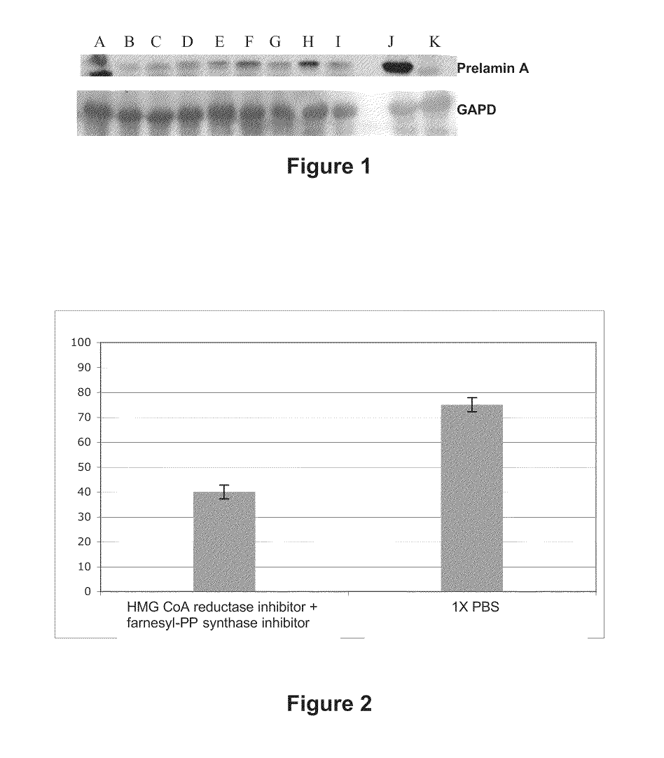

### Western Blot (FIG. 1)

“Normal” control fibroblasts were treated with a water-soluble statin (pravastatin P, 20 to 100 μM), and with an aminobiphosphonate (NBP zoledronate Z, 30 to 100 μM) in association (Tracks A to I, P20/Z20, P20/Z60, P20/Z100, P60/Z20, P60/Z60, P60/Z100, P100/Z20, P100/Z60 AND P100/Z100, respectively). The Western blot shows the “appearance” of a band corresponding to the size of the non-mature (non-truncated) prelamin A in relation to the increase in concentration of the two molecules, which confirms that farnesylation is necessary to the maturation of lamin A. This result shows that blocking the synthesis of farnesyl-PP at two points of the metabolic pathway is more effective than blocking at a single point, in terms of inhibiting the farnesylation of the prelamin A, at least ex-vivo.

### Dose- and Time-Response in Immunohistochemistry (FIG. 2)

Dose-response and time-response curves made it possible to determine maximum effectiveness by measuring 2 parameters on healthy control cells, on the one hand, and then on HGPS patient cells.

The most effective pravastatin (water-soluble)/zoledronate (NBP) combination was obtained for a 1 μM administration of pravastatin for 24 hours, and of zoledronate for 12 hours on healthy cells. No toxicity was observed. Using the same administration protocol, the number of “deformed” nuclei dropped by 75 to 40% on the HGPS cells (cells with nuclear anomalies). The rate of prelamin A obtained from healthy cells was measured. This measurement showed that this rate is maximum.

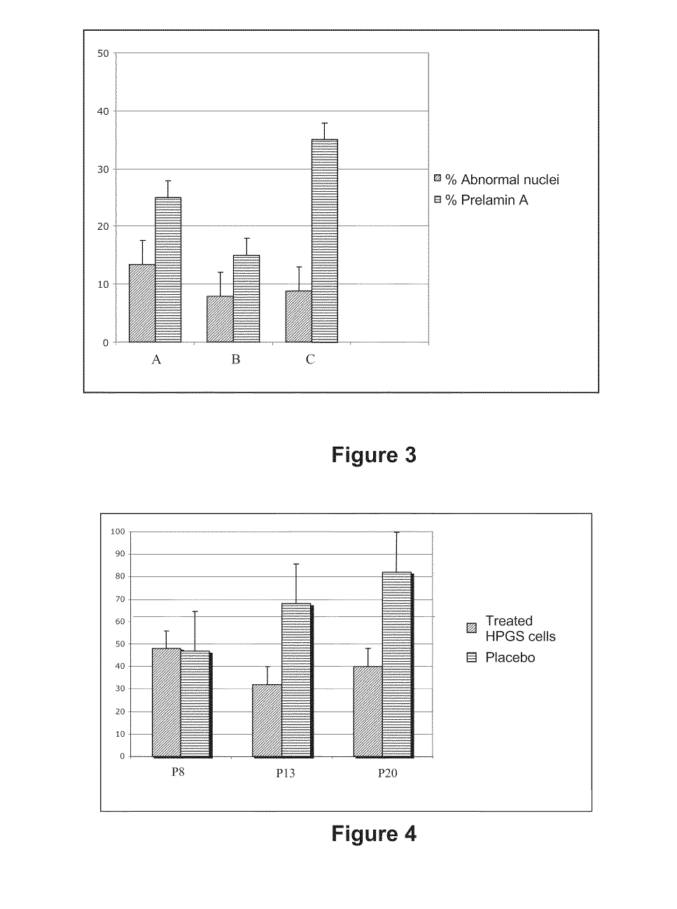

### Effect of the Immunohistochemistry Treatment (FIG. 3)

The combined action of the pravastatin and zoledronate: 100 μM pravastatin for 12 hours, 20 μM zoledronate for 6 hours shows improved efficacy, since the rate of prelamin A produced in treated healthy cells (estimated to be 35%) is much higher in combination than if the molecules are added singly (25 and 15%, respectively). In addition, the rate of deformed nuclei (a sign of toxicity in healthy cells) is minimal (less than 10%, and lower than it is for cells treated with pravastatin alone (approximately 12%).

### Immunohistochemical Effect on Aged Cells (FIG. 4)

Based on the number of “passages” (number of subcultures of the cells), and therefore based on the age of the cells, the proportion of abnormal cells increases. This characteristic is typical of untreated HGPS cells. If the HGPS cells are treated with 1 μM pravastatin for 24 hours and with 1 μM zoledronate for 12 hours, this proportion is maintained, and even decreases slightly (less than 40% versus more than 80% in cells treated with a placebo).

### Conclusion

The pravastatin/zoledronate combination is effective at doses for which almost no effect is observed when the molecules are administered separately.

The physiological effect of blocking the prenylation pathway is therefore obtained with doses much lower than those used in a single treatment, in published articles concerning cell cultures (Kusuyama et al., 2006 (27), 10 μM of pravastatin alone on vascular cell progenitors; Flint et al., 1997 (13), 25 μM of pravastatin on neonatal rat muscle cells).

### Example 2

### Effect of a Cosmetic Composition According to the Invention Including a Water-Soluble Inhibitor of hydroxymethylgultaryl-coenzyme A (HMG-CoA) Reductase and an Inhibitor of farnesyl pyrophosphate synthase on the Division of Aged Human Fibroblasts and on Young Human Fibroblasts

### Subject Matter of the Example

In this example, evaluation of the in vitro effect of a water-soluble or liposoluble inhibitor of hydroxymethylgultaryl-coenzyme A (HMG-CoA) reductase and an inhibitor of farnesyl pyrophosphate synthase on the rate of cell division (mitotic index) of fibroblasts was measured. A comparison of the effect of the composition on aged human fibroblasts in comparison with young human fibroblasts was likewise carried out. The number of active ingredients in this experiment is four, the products having been used in combinations of two. The active ingredients are:

A1: Zolendronate

A2: Alendronate

B1: Pravastatin

B2: Simvastatin

The particular combinations used in this example are: A1B1, A1B2, A2B1, A2B2.

### Protocol

In this example, two batches of fibroblasts, aged fibroblasts (lot No. 9052) and young ones (lot No. 7080) were cultured in an RPMI (Invitrogen) medium containing 10% antibiotic-free foetal calf serum, for 24 hours after trypsinization of supplied dishes.

The various active ingredients were added at a final concentration of 1 μM each for 24 hours (a 1,000-fold dilution of a stock solution in water for compounds A1, A2 and B1, or in 100% ethanol for compound B2 was carried out).

The mitotic index was evaluated by incorporation of Bromo-deoxy-uridine (BrdU) over a 45-minute period, after 24 hours of incubating the cells with one of the active ingredient combinations. Immunohistochemical analysis enables cells in DNA synthesis phase to be revealed (predivision cells). The nuclei (genetic material) were stained by incorporating di-amino phenyl indol (DAPI).

The capturing of 6 microscopic fields (OLYMPUS IX 70) enabled the mitotic index to be measured via image analysis (OLYMPUS AnalySIS). The mitotic index corresponds to the ration of the number of nuclei having incorporated the BrdU to the number of nuclei having incorporated the DAPI. A mean index is calculated statistically with a standard deviation of between 0.005 and 0.061.

A two-tailed T-test enabled measurement of the statistical significance of the results obtained.

### Results

### Overall Visual Observation of the Cells

These results show a very low dividing capability of the aged fibroblasts in the absence of any treatment, prior to the study.

The young fibroblasts showed a greater dividing capability than that of the aged fibroblasts. The dividing capability of the aged fibroblasts was less than 5%, whereas the dividing capability of the young fibroblasts was 15.6%. The difference in dividing capability between the untreated aged fibroblasts and the untreated young fibroblasts was therefore equal to 3.

When carrying out this example, not toxicity was visually observed after 24 hours of incubating the fibroblasts with tested combinations of active ingredients.

When carrying out this example, no toxic effect of the ethanol (final 0.1%) was observed after 24 hours of incubation.

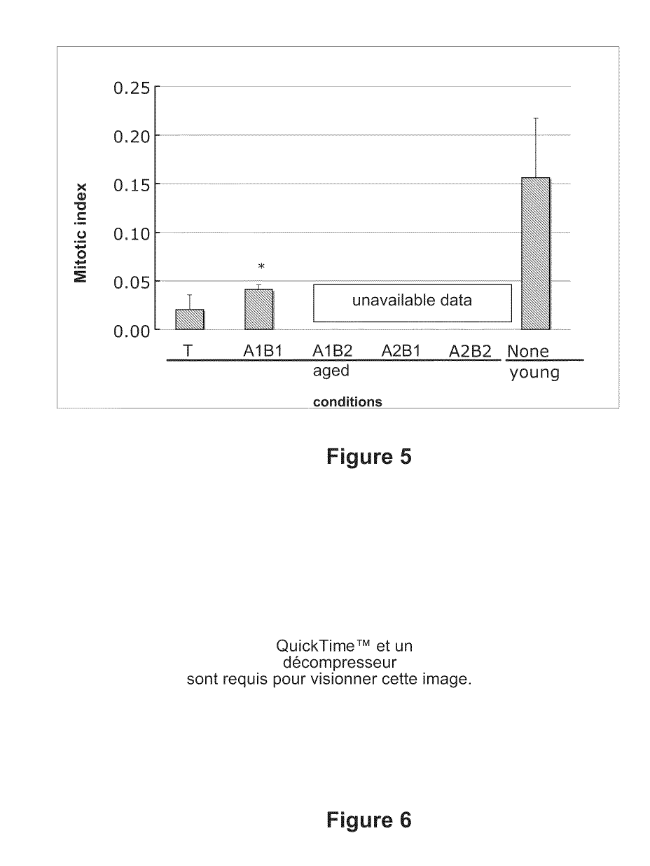

### Evaluation of the Mitotic Indicies (FIG. 5)

Generally speaking, the number of aged fibroblasts without any DNA synthesis phase treatment was extremely small: less than 5% (see FIG. 5, column 1).

The mitotic index was not very high either for the young fibroblasts: of the order of 15% (see FIG. 5, column 6).

By comparison with the control aged fibroblasts without any treatment, the control fibroblasts exposed to ethanol (0.1% -24 hours), do not show any significant difference (p=0.11, n=6) in the mitotic index thereof. The values were then consolidated (Control, n=12).

The results shown in FIG. 5, column 2 show an activating effect of A1B1=Zolendronate-Pravastatin on the mitotic index of the aged fibroblasts in comparison with the control (stimulation by a factor of 2 at a maximum) (p, 0.991, n≧6).

This example thus shows that application of the Zolendronate-Pravastatin combination has an activating effect on the cell division of fibroblasts of an elderly subject.

### EXAMPLE 3

### Effect of the Association of a Water-Soluble Inhibitor of Hydroxymethylgultaryl-Coenzyme A (HMG-CoA) Reductase and an Inhibitor of Farnesyl Pyrophosphate Synthase on a Mouse Model Having a Progeroid Syndrome

The Zmpste24^{-/-} KO mice used here are those described in the referenced article by Varela et al., 2005 (49). Proof of the efficacy of associating the 2 molecules (pravastatin and zoledronate) was furnished in cooperation with a Spanish laboratory (Pr C. Lopez-Otin). The efficacy is obtained at combined doses which have not effect when the products are used separated, thereby demonstrating a synergistic effect.

The 2 molecules (zolendronic acid (Zometa trademark)) 100 μg/kg/day and pravastatin 100 mg/kg/day) diluted with 1× PBS and injected intraperitoneally, daily, into 1-month old mice and up until the death thereof. The controls are wild mice of the same range treated with 1× PBS only.

The survival of the treated mice was greatly improved, and was maximum, in particular, for the females, with an average increase in life span of approximately 80%. The clinical symptoms of the disease were considerably reduced in comparison with the individual treated with the PBS alone. In particular, an effect of the treatment on the skin and the regrowth of the hair of these mice were observed, in comparison with the mice treated with the PBS, which showed large hairless regions.

### EXAMPLE 4

### Cosmetic Compositions According to the Invention Including a Water-Soluble Inhibitor of Hydroxymethylgultaryl-Coenzyme A (HMG-CoA) Reductase and an Inhibitor of Farnesyl Pyrophosphate Synthase

Composition 1

| Ingredients | Percentage |
|---|---|
| Pravastatin | 0.075 |
| Alendronic Acid | 0.025 |
| Denatured Alcohol | 15.00 |
| PEG-8 Dimethicone | 5.000 |
| Glyceryl Polymethacrylate | 4.950 |
| Glycerin | 3.000 |
| PEG-400 | 3.000 |
| Butylene Glycol | 1.070 |
| Phenoxyethanol | 0.430 |
| Perfume (Fragrance) | 0.350 |
| Copolymer Acrylates/C10-30 Alkyl | 0.320 |
| Acrylate | |
| PPG-26-Buteth-26 | 0.220 |
| Chlorphenesin | 0.200 |
| Tromethamine | 0.170 |
| PEG-40 hydrogenated castor oil | 0.140 |
| Titanium dioxide, Mica, Silica | 0.300 |
| Methylparaben | 0.120 |
| Propylene Glycol | 0.050 |
| Laureth-3 | 0.050 |
| Ethylparaben | 0.024 |
| Butylparaben | 0.024 |
| Hydroxyethylcellulose | 0.020 |
| Propylparaben | 0.015 |
| Sodium Hyaluronate | 0.005 |
| Water | QS 100 |

Composition 2

| Ingredients | Percentage |
|---|---|
| Pravastatin | 0.075 |
| Alendronic Acid | 0.025 |
| Glycerin | 5.000 |
| Beheneth-25 | 5.000 |
| Dicaprylyl Carbonate | 4.000 |
| Octyldodecanol | 3.000 |
| Cetearyl alcohol | 3.000 |
| Gylceryl Polymethacrylate | 2.970 |
| Butylene Glycol | 2.010 |
| Dimethicone | 2.000 |
| Decyl Oleate | 2.000 |
| Sweet almond oil | 2.000 |
| Cetyl Palmitate | 2.000 |
| Polymethylmethacrylate | 1.000 |
| Phenoxyethanol | 0.430 |
| Butylparaben | 0.424 |
| Sodium Polyacrylate | 0.300 |
| Copolymer Acrylates/C10-30 Alkyl | 0.300 |
| Acrylate | |
| Chlorphenesin | 0.300 |
| Tocopheryl Acetate | 0.250 |
| Perfume (Fragrance) | 0.200 |
| Laureth-3 | 0.150 |
| Methylparaben | 0.120 |
| Disodium EDTA | 0.100 |
| Xanthan Gum | 0.100 |
| Sodium Hydroxide | 0.089 |
| Hydroxyethylcellulose | 0.060 |
| PEG-8 (polyethylene glycol) | 0.049 |
| Propylene Glycol | 0.030 |
| Tocopherol | 0.025 |
| Ethylparaben | 0.024 |
| Propylparaben | 0.015 |
| Isobutylparaben | 0.010 |
| Ascorbyl Palmitate | 0.004 |
| Ascorbic Acid | 0.0008 |
| Citric Acid | 0.0008 |
| Water | QS 100 |

Composition 3

| Ingredients | Percentage |
|---|---|
| Caprylic/Capric Triglyceride | 5.000 |
| Glyceryl Polymethacrylate | 4.950 |
| Steareth-21 | 3.000 |
| Glycerin | 3.000 |
| Polymethyl Methacrylate | 3.000 |
| Butylene Glycol | 2.010 |
| Steareth-2 | 2.000 |
| Cyclopentasiloxane | 1.505 |
| Butyrospermum Parkii (shea butter) | 1.000 |
| Sweet almond oil | 1.000 |
| Laureth-3 | 0.750 |
| Sodium PCA | 0.523 |
| Phenoxyethanol | 0.430 |
| Carbomer | 0.300 |
| Tocopherol Acetate | 0.250 |
| Boron Nitride | 0.250 |
| Chlorphenesin | 0.250 |
| Dimethicone Copolymer | 0.245 |
| Perfume (Fragrance) | 0.200 |
| Polyacrylamide | 0.197 |
| Methylparaben | 0.120 |
| C14-14 Isoparaffin | 0.100 |
| Disodium EDTA | 0.100 |
| Sodium Hydroxide | 0.0875 |
| Hydroxyethylcellulose | 0.060 |
| Propylene Glycol | 0.050 |
| Laureth-7 | 0.025 |
| PEG-8 (Polyethylene glycol), Tocopheral, | 0.080 |
| Ascorbyl Palmitate, Ascorbic Acid, | |
| Citric Acid | |
| Ethylparaben | 0.024 |
| Butylparaben | 0.024 |
| Propylparaben | 0.015 |
| C.I 42090 (Blue 1) | 0.0002 |
| Pravastatin | 0.075 |
| Alendronic Acid | 0.025 |
| Water | QS 100 |

Composition 4

| Ingredients | Percentage |
|---|---|
| Caprylic/Capric Triglyceride | 5.000 |
| Glyceryl Polymethacrylate | 4.950 |
| Steareth-21 | 3.000 |
| Glycerin | 3.000 |
| Polymethyl Methacrylate | 3.000 |
| Butylene Glycol | 2.010 |
| Steareth-2 | 2.000 |
| Cyclopentasiloxan | 1.505 |
| Butyrospermum Parkii (shea butter) | 1.000 |
| Sweet almond oil | 1.000 |
| Laureth-3 | 0.750 |
| Sodium PCA | 0.523 |
| Phenoxyethanol | 0.430 |
| Carbomer | 0.300 |
| Tocopherol Acetate | 0.250 |
| Boron Nitride | 0.250 |
| Chlorphenesin | 0.250 |
| Dimethicone Copolymer | 0.245 |
| Perfume (Fragrance) | 0.200 |
| Polyacrylamide | 0.197 |
| Methylparaben | 0.120 |
| C13-14 Isoparaffin | 0.100 |
| Disodium EDTA | 0.100 |
| Hydroxyethylcellulose | 0.060 |
| Propylene Glycol | 0.050 |
| Laureth-7 | 0.025 |
| PEG-8, Tocopherol, Ascorbyl Palmitate, | 0.080 |
| Ascorbic Acid, Citric Acid | |
| Ethylparaben | 0.024 |
| Butylparaben | 0.024 |
| Propylparaben | 0.015 |
| C.I 42090 (Blue 1) | 0.0002 |
| Pravastatin | 0.095 |
| Zolendronic Acid | 0.005 |
| Water | QS 100 |

Composition 5

| Ingredients | Percentage |
|---|---|
| Denatured Alcohol | 15.000 |
| PEG-8 Dimethicone | 5.000 |
| Glyceryl Polymethacrylate | 4.950 |
| Glycerin | 3.000 |
| PEG-400 | 3.000 |
| Butylene Glycol | 1.070 |
| Phenoxyethanol | 0.430 |
| Perfume (Fragrance) | 0.350 |
| Copolymer Acrylates/C10-30 Alkyl | 0.320 |
| Acrylate | |
| PPG-26-Buteth-26 | 0.220 |
| Chlorphenesin | 0.200 |
| Tromethamine | 0.170 |
| PEG-40 hydrogenated castor oil | 0.140 |
| Titanium Dioxide, Mica, Silica | 0.300 |
| Methylparaben | 0.120 |
| Propylene Glycol | 0.050 |
| Ethylparaben | 0.024 |
| Butylparaben | 0.024 |
| Hydroxyethylcellulose | 0.020 |
| Propylparaben | 0.015 |
| Sodium Hyaluronate | 0.005 |
| Pravastatin | 0.095 |
| Zolendronic Acid | 0.005 |
| Water | QS 100 |

Composition 6

| Ingredients | Percentage |
|---|---|
| Glycerin | 5.000 |
| Beheneth-25 | 5.000 |
| Dicaprylyl Carbonate | 4.000 |
| Octyldodecanol | 3.000 |
| Cetearyl Alcohol | 3.000 |
| Gylceryl Polymethacrylate | 2.970 |
| Butylene Glycol | 2.010 |
| Dimethicone | 2.000 |
| Decyl Oleate | 2.000 |
| Sweet almond oil | 2.000 |
| Cetyl Palmitate | 2.000 |
| Polymethylmethacrylate | 1.000 |
| Phenoxyethanol | 0.430 |
| Butylparaben | 0.424 |
| Sodium Polyacrylate | 0.300 |
| Acrylates/C10-30 Alkyl Acrylate | 0.300 |
| Copolymer | |
| Chlorphenesin | 0.300 |
| Tocopheryl Acetate | 0.250 |
| Perfume (Fragrance) | 0.200 |
| Laureth-3 | 0.150 |
| Methylparaben | 0.120 |
| Disodium EDTA | 0.100 |
| Xanthan Gum | 0.100 |
| Sodium Hydroxide | 0.089 |
| Hydroxyethylcellulose | 0.060 |
| PEG-8 (Polyethylene Glycol) | 0.049 |
| Propylene Glycol | 0.030 |
| Tocopherol | 0.025 |
| Ethylparaben | 0.024 |
| Propylparaben | 0.015 |
| Isobutylparaben | 0.010 |
| Ascorbyl Palmitate | 0.004 |
| Ascorbic Acid | 0.0008 |
| Citric Acid | 0.0008 |
| Pravastatin | 0.095 |
| Zolendronic Acid | 0.005 |
| Water | QS 100 |

Composition 7

| Ingredients | Percentage |
|---|---|
| Demineralised water | 86.678% |
| Cetiol A (registered trademark) (hexyl | 5.000% |
| Laurate) | |
| Glycerin Codex | 3.000% |
| Simulsol 165 (registered trademark) | 1.500% |
| (glyceryl stearate) | |
| Montanov 14 (trademark) (myristyl | 1.000% |
| alcohol and myristyl glucoside) | |
| Phenonip (registered trademark) | 0.600% |
| (phenoxyethanol, methylparaben, | |
| butylparaben, ethylparaben, | |
| propylparaben) | |
| 10% aqueous NaOH solution | 0.512% |
| Covabsorb (registered trademark) | 0.500% |
| (ethylhexyl Methoxycinnamate, Butyl | |
| methoxydibenzoylmethane, ethylhexyl | |
| Salicylate, PPG-26-Buteth-26, | |
| hydrogenated Polyethyleneglycol G-40, | |
| Castor oil | |
| Pravastatin | 0.500% |
| Carbopol (unregistered mark) (prop-2- | 0.300% |
| enoic acid) | |
| Chlorphenesin | 0.250% |
| Sepigel 305 (trademark) (Polyacrylamide | 0.100% |
| and Isoparaffin at C13-14 and Laureth-7) | |
| Alendronate | 0.060% |
| | 100.000% |

Composition 8

| | |
|---|---|
| Demineralised water | 87.218% |
| Cetiol A (registered trademark) (hexyl | 5.000% |
| Laurate) | |
| Glycerin Codex | 3.000% |
| Simulsol 165 (registered trademark) | 1.500% |
| (glyceryl stearate) | |
| Montanov 14 (trademark) (myristyl | 1.000% |
| alcohol and myristyl glucoside) | |
| Phenonip (registered trademark) | 0.600% |
| (phenoxyethanol, methylparaben, | |
| butylparaben, ethylparaben, | |
| propylparaben) | |
| 10% aqueous NaOH solution | 0.512% |
| Covabsorb (registered trademark) | 0.500% |
| (ethylhexyl Methoxycinnamate, Butyl | |
| methoxydibenzoylmethane, ethylhexyl | |
| Salicylate, PPG-26-Buteth-26, | |
| hydrogenated Polyethyleneglycol G-40, | |
| Castor oil | |
| Carbopol (unregistered mark) (prop-2- | 0.300% |
| enoic acid) | |
| Chlorphenesin | 0.250% |
| Sepigel 305 (trademark) (Polyacrylamide | 0.100% |
| and Isoparaffin at C13-14 and Laureth-7) | |
| Pravastatin | 0.010% |
| Alendronate | 0.010% |
| | 100.00% |

### EXAMPLE 5

### Skin Irritation Tests on Reconstructed Skin Using Cosmetic Compositions According to the Invention Including a Water-Soluble Inhibitor of Hydroxymethylgultaryl-Coenzyme A (HMG-CoA) Reductase and an Inhibitor of Farnesyl Pyrophosphate Synthase

In this example, cosmetic compositions including a water-soluble inhibitor of hydroxymethylgultaryl-coenzyme A (HMG-CoA) reductase (a statin: pravastatin) and an inhibitor of farnesyl pyrophosphate synthase, zolendronic acid or alendronic acid, are applied to human epidermis reconstructed in vitro. The dimension of the dermal layer is 0.5 cm². Various quantities of the cosmetic composition are applied, ranging between 30 to 50 mg.

The cell viability is determined by an “MTT” assay. The “MTT” assay is a colorimetric assay based on the quantification of mitochondrial dehydrogenases, which are enzymes indicating cell viability. This assay is carried out at two different application times: 24 and 72 hours.

Positive controls using Sodium Dodecyl Sulfate (SDS) and negative controls are likewise carried out.

### EXAMPLE 6

### Acute Eye Irritation Tests Using Cosmetic Compositions According to the Invention, Including a Water-Soluble Inhibitor of Hydroxymethylgultaryl-Coenzyme A (HMG-CoA) Reductase and an Inhibitor of Farnesyl Pyrophosphate Synthase

In this example, the tests are carried out according to “good laboratory practices” in accordance with regulatory requirements, the Order of Aug. 10, 2004 and Article L5131-5 of the Public Health Code.

Evaluation of the cytotoxicity is carried out using the technique of salting out neutral red on cornea cells of the SIRC line.

### EXAMPLE 7

### Effects of the Combination of a Water-Soluble Hydroxymethylglutaryl-Coenzyme A (HMG-CoA) Reductase and an Inhibitor of Farnesylpyrophosphate Synthase on Ex-vivo Extracts of Human Skin

### Protocol

In this example, the tests were carried out on skin derived from a donor of approximately 60 years old. A preparation of 21 explants of human skin was made and kept alive in a BEM medium (BIO-EC's Explants Medium). The explants were divided into 3 batches of six explants and one control batch T0 of 3 explants, as follows:
T0 Plastics Control: 3 explants
T Positive control: 6 explants
P Explants treated with the composition of the invention: 6 explants

### Treatment

The treatment was carried out on different days, on the first day (J0), two hours after preparation of the explants, then on D+1, D+2 days, D+4 days, D+6 days, D+8 days and D10+days.

The products were applied to the explants as follows:
T: the explants do not receive any treatment,
R: the explants each receive 1 mg of the positive control (retinol cream) on D0, D+2 and D+4.
P1: the explants each receive 2 mg of the product P at each treatment time.

The treatment was carried out via topical application of the composition of the invention. The composition was next distributed over the entire surface of the explant, using a spatula. Half of the culture medium was replenished every two days and the explants were kept alive at 37° C. in a moist atmosphere enriched with 5% CO₂.

### Sampling for Histology

At D0, the 3 explants of lot T0 were sampled.

At D+6 days and D+11 days, 3 explants from each lot were sampled. The samples were cut in two, one half was fixed in formol and the other half was frozen at −80° C., according to the BIO-EC “P006-PPEH” procedure.

### Histological Study

After 24 hours of fixing in formol, the samples were dehydrated, impregnated and coated with paraffin. 5-μm sections were made for morphological observation.

### First Step: Morphological Study

The morphological study of the epidermal and dermal structures was carried out on sections coloured with Masson's trichrome, a variant of Goldner's.

### Second Step:

Immunolabelling with Kl67:

Immunolabelling of the cells in mitosis was carried out on frozen sections with the anti-Kl 67 polyclonal antibody (Novo Castra) developed in DAB. The positive cells are counted over the entire epidermal length and the averages normalized to the number of cells labelled per cm.

Immunolabelling with collagen I:

Immunolabelling with collagen I was carried out on frozen sections with the anti-collagen I polyclonal antibody developed in FITC. The nuclei were counter-coloured with propidium iodide.

Immunolabelling with collagen III:

Immunolabelling with collagen III was carried out on frozen sections with the anti-collagen III polyclonal antibody developed in DAB. The nuclei were counter-coloured with Masson's hemalum.

Immunolabelling with collagen IV:

Immunolabelling with collagen IV was carried out on frozen sections with the anti-collagen IV polyclonal antibody (Cliniscience) developed in FITC. The nuclei were counter-coloured with propidium iodide.

Immunolabelling with collagen VII:

Immunolabelling with collagen VII was carried out on frozen sections with the anti-collagen VII monoclonal antibody developed in FITC. The nuclei were counter-coloured with propidium iodide.

Immunolabelling with PECAM1:

The endothelial cells were viewed by immunolabelling with PECAM-1, which was carried out on frozen sections with the anti-PECAM1 monoclonal antibody developed in Fast-red.

### EXAMPLE 8

### Effect of the Combination of a Water-Soluble Hydroxymethylglutaryl-Coenzyme A (HMG-CoA) Reductase and the Inhibitor of Farnesylpyrophosphate Synthase on in-vitro Cultures of Constitutive Skin Cells.

This example was carried out with the same combinations of active ingredients as in example 2 above. These various combinations of active ingredients were used in vitro in order to evaluate the effect thereof on physiological parameters involved in skin ageing.

The combinations used in this example are A1B1, A1B2, A2B1, A2B2, respectively. These four combinations were tested at several concentrations, the experiments being conducted in triplicate (which represents at least 36 experimental points).

The concentrations of the 4 combinations are proposed by the applicant and, therefore, at this design stage, no in vitro cytotoxicity is anticipated. The experiment was conducted on fibroblast cultures as presented in example 1. This test was likewise applied to keratinocyte cultures. The following parameters were examined for the 4 combinations of active ingredients at the concentrations indicated.

### Measurement of the Mitotic Index

Measurement of the remodelling of the extracellular matrix via contraction of collagen lattices.
Measurement of the distribution of genomic DNA after UVB irradiation (photoinduced stress approximating the sunbathing conditions).

Measurement of the mitotic index was carried out after exposing the cells to the active ingredients single time. The index was estimated via image analysis counting of the cell nuclei having incorporated a thymidine analogue rendered fluorescent out of the total number of nuclei. Several fields were analysed. Photos were archived for iconographic purposes.

Remodelling of the extracellular matrix induced by the fibroblasts exposed to the active ingredients was evaluated by incorporating these cells into collagen lattices and by quantifying the capability thereof to retract these lattices. Evaluation of the retracted surface yielded a remodelling index. Photos were archived for iconography purposes.

Measurement of the distribution of the genomic DNA was carried out after irradiating the cells with a dose of UVB mimicking the conditions of a sun burn. In a first phase, it was anticipated to evaluate the effect of the active ingredients during the follow-up DNA repair in a 3-phase kinetics operation. Quantification was carried out via detection and image analysis of the cyclobutene pyriminide dimers induced by UVB irradiation using an immunohistochemical technique.

Photos are archived for iconography.

### EXAMPLE 9

### A Treatment Combining a Statin and an Aminobiphosphonate Increases the Lifespan of a Mouse Model Reproducing a Human Syndrome of Premature Ageing

This example is likewise published in Varela et al., Nature Medicine 2008, 7, 767 (55).

Material and methods

### Mice:

The production of the Zmpste24^{-/-} and Lmna^{—/-} mice was described (Pendas al., 2002 (38); Sullivan al., 1999 (56)). Computerized microtomography of the bones of the mice was carried out using the micro-CT SkyScan 1172 system (SkyScan—trademark)). All of the experiments on the mice complied with the rules enacted by the Committee on Animal Experimentation of the University of Oviédo (Spain). Pravastatin (100 mg/kg/day) and zoledronate (100 mg/kg/day) diluted in PBS were administered to the mice every day. The mice receiving the pravastatin-zoledronate treatment or the control mice receiving only PBS did not have any apparent injury or stress.

### Cell Culture

The dermal fibroblasts of a control subject (GM00038) and of patients afflicted with progeria and carriers of the G608G mutation (AG11498 and AG01972) were obtained from the Coriell Cell Repository. The HeLa cells came from the American Type Culture Collection. The cells were cultivated in DMEM (Gibco) supplemented with 10% FBS (Gibco) and 1% antibiotic-antimycotic (Gibco). The fibroblasts came from 12-day old mice tails (Varela et al., 2005). The concentration and the length of the treatment with the statin and the aminobiphosphonate are indicated in the legend to the figures. During the combined treatment with statin+aminobiphosphonate in the presence of farnesol and/or geranylgeraniol, 1 mM of pravastatin and 1 mM of zoledronate were added to the culture medium with increasing concentrations of farnesol and/or geranylgeraniol. The percentage of abnormal nuclei was measured 48 hours after the start of the treatment.

### Immunocytochemistry

The fibroblasts were cultivated in Lab Tek (Nalgen Nunc International) chambers, washed in PBS, and then fixed in 4% paraformaldehyde for 15 min. The cells were dehydrated in ethanol baths of increasing concentration and permeabilized for 5 min at 25° C. in PBS containing Triton X-100 (0.5%), 5% serum (goat or rabbit). The slides were pre-incubated at 25° C. in PBS for 5 min.

The dilution of primary antibodies was 1:100 for the polyclonal goat anti-prelamin A antibody Sc-6214, Santa Cruz Biotechnologies), 1:40 for the anti-lamin A/C antibody (4A7 supplied by G. Morris), 1:200 for the anti-calreticulin rabbit antibody (Stressgen) and 1:100 for the anti-lamin B1 antibody (Abcam). After washing in PBS, the slides were incubated for 1 hour at 25° C. with the secondary antibodies diluted in the incubating solution. The dilution of the secondary antibodies was as follows: 1:100 for the FITC-coupled anti-mouse donkey IgG (Jackson ImmunoResearch), 1:400 for the Alexa 488-coupled anti-goat donkey IgG (Molecular Probes) and 1:100 for the tetramethylrhodamine isothiocyanate-coupled anti-rabbit donkey IgG (Sigma). The cells were then washed, the nuclei coloured for 15 min at 25° C. with DAPI 9100 ng/ml, Sigma-Aldrich), and finally washed 3 times with PBS containing 0.5% Tween 20. The slides were mounted in Vectashield (Vector). Digital images were recorded with a Leica DMR microscope equipped with a CoolSnap camera or with a Leica TCS SP5 confocal microscope. The nuclei were observed in cells after labelling of the lamin A/C. More than 100 nuclei were analysed in control fibroblasts for each of the treatments. The number of cell nuclei of patients afflicted with progeria which was analysed was 250 (passage 8), 198 (passage 13) and 150 (passage 20).

### X-ray Irradiation and Study of the Phosphorylated Histone H2AX

The patients of progeria patients and the 1BR3 control cells were irradiated with Philips X-ray equipment. The X-ray beam was produced by a tungsten anode subjected to a voltage of 200 kV at an intensity of 20 mA, with a copper filter measuring 0.1 mm in diameter. The dose rate was 1.243 Gy/min. The phophorylated histone H2AX was detected with antibodies recognizing specifically the phosphorylated serine 139 (Upstate Biotechnology-Euromedex, Mundolsheim, France), at the dilution of 1:800, and FITC-conjugated anti-mouse antibodies (1:100, Sigma-Aldrich). The number of double-stranded breaks (DSB) in relation to the repair time was adjusted using the formula Nt=N0 (1/1+βt)^{α}, where α and β are adjustable parameters and Nt and N0 are the number of DSB at time t and time 0.

### Western Blot

The cells were homogeneous in the following medium: 50 mM Tris (ph 7.4), 150 mM NaCl, 1% NP-40, 50 mM NaF, 1 mM dithiothreitol, 2 mg/ml pepstatin A, in the presence of protease inhibitors (Complete inhibitor cocktail, Roche) and phosphotase inhibitors (Phosphotase Inhibitor Cocktails I and II, Sigma). After electrophoresis, the proteins were transferred onto nitrocellulose membranes blocked with 5% nonfat milk powder, using the TBS-T buffer (20 mM Tris pH 7.4, 150 mM NaCl and 0.05% Tween-20), and incubated for 1 hour with either a specific anti-lamin A/C antibody (4A7, 1:500) or a specific anti-lamin A antibody (C20, Santa Cruz Biotechnology, 1:250) or an actin anti-beta antibody (A5441, Sigma, 1:5000). The antibodies were diluted in TBS-T containing 3% nonfat milk powder. The blots were then incubated with a peroxidise-coupled antibody (anti-mouse or anti-rabbit goat, Jackson ImmunoResearch) in TBS-T containing 1.5% nonfat milk powder, then washed, and finally developed via chemiluminescence (Immobilon Western chemiluminescent HRP substrate, Millipore—trademark).

### Mass Spectrometry Analysis

The Zmpste24^{-/-} and control mouse fibroblasts, as well as the lmyphoblastoid cells of progeria patients, were homogenised, the nuclei isolated by ultracentrifugation and the nuclear proteins obtained as described in Blobel and Potter, V. R. Nuclei from rat liver: isolation method that combines purity with high yield, Science 154, 1662-1665, 1966. The proteins of the nuclear lamina were separated by SDS-PAGE electrophoresis, and the bands containing the lamin A, the prelamin A and the progerin were excised. The gel fragments were washed twice with 180 ml of an ammonium bicarbonate/acetonitrile mixture (70:30, 25 mM), dried (15 min, 90° C.) and digested (1 hour, 60° C.) with trypsin (12 ng/ml, Promega) in 25 mM ammonium bicarbonate. In a typical experiment, 1 ml of CHCA (a-cyano-4-hydroxycinnamic acid, Waters) was placed in a MALDI-ToF spectrometer. Once dry, 1 ml of the peptide solution and 1 ml of the matrix (CHCA) were placed in the spectrometer equipped with a laser source (Voyager-DE STR (trademark), Applied Biosystems). The data collected from 200 laser shots produced spectra that were analysed by the Data Explorer program (Version 4.0.0.0, Applied Biosystems).

### Real-Time Quantitative PCR

The rate of expression of p53 target genes (Atf3, Gadd45g and Cdkn1a, which codes for p21) was measured using the ABI PRISM 7900HT Sequence detection system device (Applied Biosystems).

### Statistical Analysis

The difference between the various groups of treated or untreated mice and cells was analysed using the t-test. The calculations were made with the Microsoft Excel program. The data was expressed as a mean±standard error of the mean (SEM).

### Results

HeLa cells were cultivated in the presence of inhibitors of farnesyltransferase (FTI-277, Sigma-Aldrich) and or of type I geranylgeranyltransferase inhibitors (GGTI-2147, Sigma-Aldrich) at the indicated concentrations. Only the combination of both inhibitors leads to the accumulation of a significant quantity of non-prenylated prelamin A in the cells, in comparison with the effect of each of the inhibitors used alone.

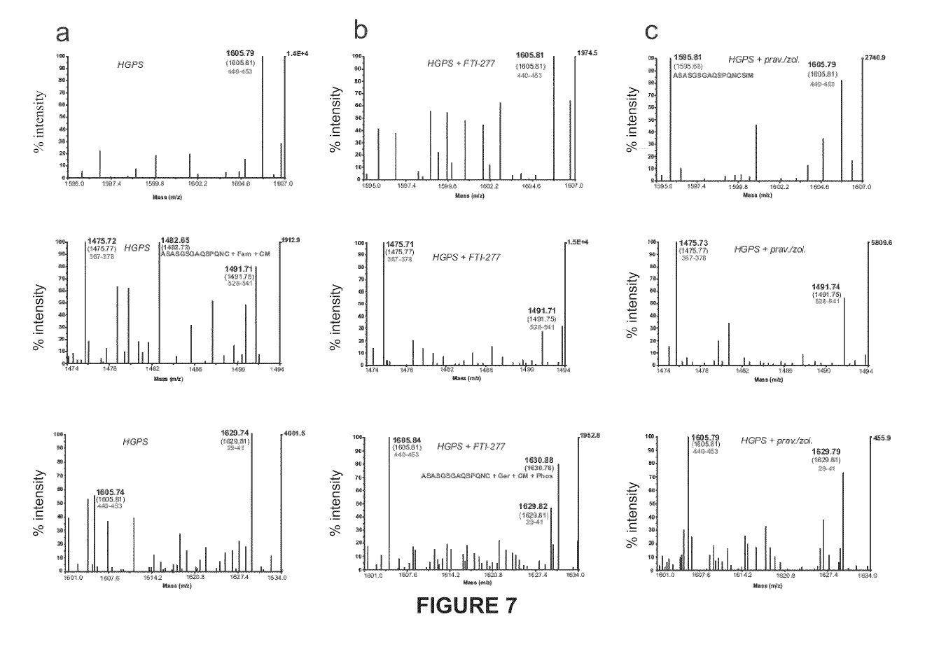

These results are shown in FIG. 6, which is a photograph of a Western Blot obtained, showing detection of the lamin A/C in HeLa cells treated with inhibitors of farnesyltransferase and/or with type I geranylgeranyltransferase inhibitors. LA=lamin A, LC=lamin C, Pre=prelamin A.

These results confirm that blocking the prenylation of prelamin A requires both the inhibition of farnesyltransferase and of type I geranylgeranyltransferase in accordance with this invention.

The Farnesyltransferase Inhibitor (FTI) Induces Compensatory Geranylgeranylation of the Progerin (in Cells of Patients Afflicted with Progeria) and in Fibroblasts of Zmpste24^{-/-} Mice.

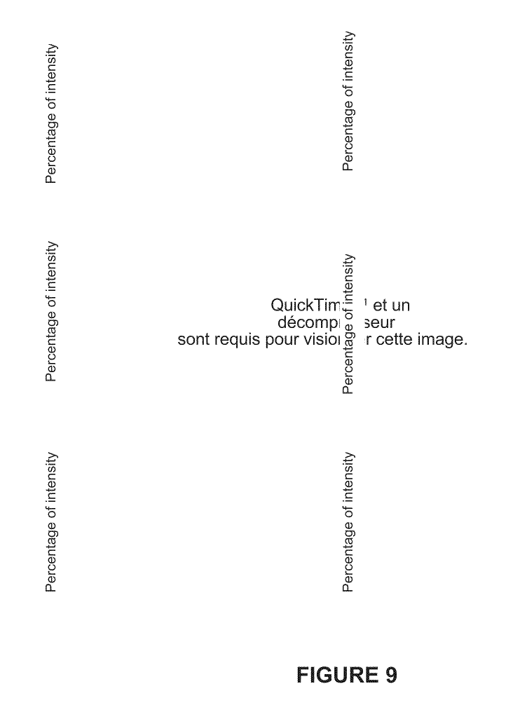

As expected, mass spectrometry analysis shows the presence of tryptic peptides of the farnesylated and carboxymethylated prelamin A in cells of Zmpste24^{-/-} mice, but not in the cells of control mice. These results are shown in FIG. 9a. The farnesylated peptide is devoid of the 3 SIM residues, which shows that Zmpste24 is not indispensable to the first cleavage during maturation of the prelamin A. A decrease in the quantity of farnesylated prelamin A was observed in the cells of mice treated with FTI. During observation of the portion of the spectrum corresponding to the geranlgeranylated peptides, no peptide derived from prelamin A could be detected in the cells of untreated Zmpste24^{-/-} mice. However, a peptide the mass of which was compatible with a geranylgeranylated fragment of prelamin A was detected after treatment with FTI. These results are shown in FIG. 9b.

In the cells of progeria patients, peptides corresponding to farnesylated and carboxymethylated progerin were detected in the absence of any treatment, as indicated in FIG. 7a. The treatment of these cells with FTI-127 caused the appearance of peptides the mass of which corresponds to that of geranylgeranylated peptides of progerin, as seen in FIG. 7b.

All of this data shows that the progerin and the prelamin A were geranylgeranylated alternately under the effects of the FTIs, and provides an explanation as to the low degree of effectiveness of the FTI treatments in murine models of progeroid syndrome.

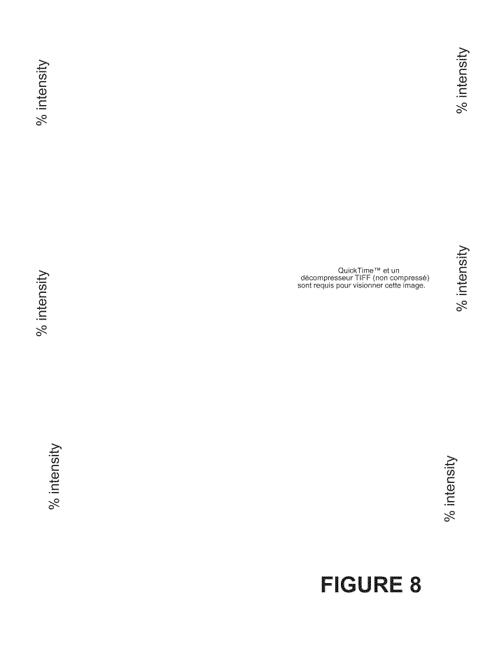

The cells of progeria patients and of Zmpste24^{-/-} mice were used to evaluate therapeutic strategies intended to prevent cross-prenylation of prelamin A and progerin. We have made the hypothesis that farnesylation of the various abnormal variants of lamin A might be inhibited by drugs acting on the biosynthesis pathway of farnesylpyrophosphate, a substrate of farnesyltransferase and precursor for geranylgeranyl pyrophosphate, a substrate of type I geranylgeranyltransferase. Therefore, we tested the effect of two drugs, a statin and an aminobiphosphonate, which are known to act on cells of progeria patients at two stages of this metabolic pathway. Mass spectrometry analysis shows that the pravastatin (statin) and zoledronate (aminobiphosphonate) combination causes the appearance of a peptide corresponding to the non-prenylated C-terminal end of progerin, a peptide which is undetectable in cells treated with FTI, while neither the farnesylated peptides or geranylgeranylated peptides were detected either, as seen in FIG. 7c). The statin+aminobiphosphonate treatment did indeed inhibit prenylation of the progerin. The same observation was made for prelamin A, as seen in FIG. 8. The non-prenylated C-terminal peptide of same was detected in cells treated with the statin+aminobiphosphonate mixture, while it was absent from the untreated cells, in which the farnesylated and carboxymethylated peptide was found. Finally, unlike the FTI, the pravastatin+zoledronate treatment does not reveal the geranylgeranylated prelamin A.

Legend to FIG. 9: Lamin A (control cells) and prelamin A (Zmpste24^{-/-} mice cells) were analysed by mass spectrometry (MALDI-ToF). a, b: portions of the spectrum corresponding to the farnesylated (a) and geranylgeranylated (b) tryptic peptides. Each of the peaks is labelled with the theoretical mass (between parentheses) of the peptide derived from the trypsin digestion of lamin A or prelamin A. The number of residues is clearly noted in blue. The peptide sequence and the mass thereof are noted in red. Farn=farnesyl; CM=carboxymetylated; Ger=geranylgeranylated.

Legend to FIG. 7: Mass spectrometry analysis of proteins extracted from the nuclear envelope of untreated cells (a), cells of progeria patients treated with FTI (2.5 mM, b) or treated with the pravastatin+zoledronate mixture (1 μM each, c). The portion of the spectra corresponding to the unmodified, farnesylated and geranylgeranylated proteins is shown at the top, centre and bottom of the figure. Each peak corresponds to the tryptic progerin peptide and is labelled with the monoisotopic mass measured in the experiment and with the theoretical mass (between parentheses). The number of amino acid residues is noted in blue. The peptide sequence and mass thereof are noted in red.

The progerin is predominantly farnesylated (F) and carboxymethylated (Cm) in the untreated cells (a, centre panel), while, under the effects of the FTIs, this peak is very low and the progerin appears to be geranylgeranylated and phosphorylated (b, bottom panel). After the pravastatin-zoledronate treatment, the unmodified progerin is the predominant form.

Legend to FIG. 8: Mass spectrometry analysis of proteins extracted from the nuclear envelope of untreated fibroblasts (a), or ones treated with the pravastatin+zoledronate mixture (1 μM each, b) derived from Zmpste24^{-/-} mice. The portion of the spectra corresponding to the unmodified, farnesylated and geranylgeranylated proteins is shown at the top, centre and bottom of the figure. Each peak corresponds to the tryptic progerin protein and is labelled with the monoisotopic mass measured in the experiment and with the theoretical mass (between parentheses). The number of the amino acid residues is noted in blue. The peptide sequence and mass thereof are noted in red.

In this figure, it is noted that the prelamin A is predominantly farnesylated (F) and carboxymethylated (Cm) in the untreated cells (a, centre panel), while the unmodified or geranylgeranylated forms are not detected. After treatment with pravastatin+zoledronate, the prenylated proteins are no longer detectable and the unmodified form of prelamin A is predominant (b, top panel).

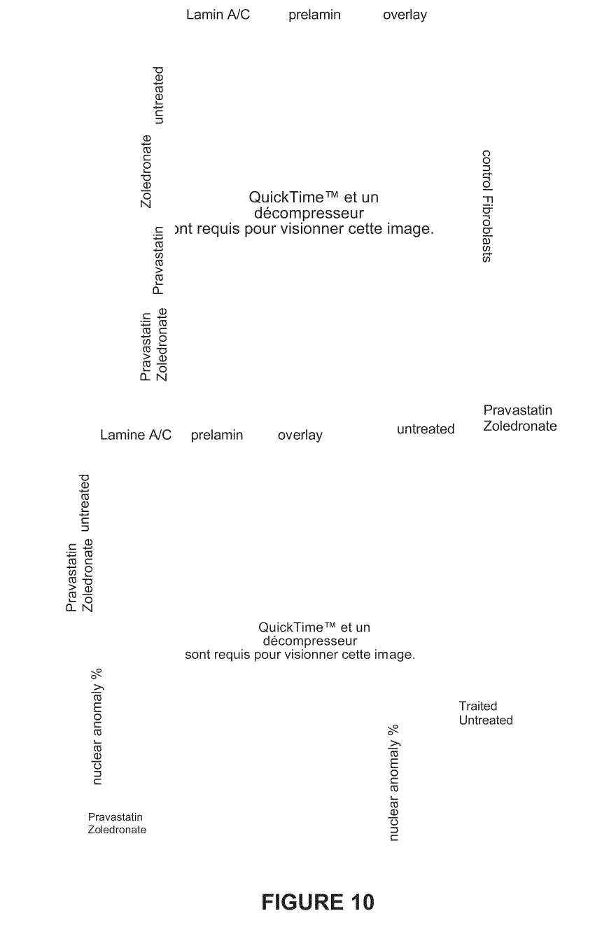

The Pravastatin+Zoledronate Treatment Corrects the Nuclear Anomalies of the Cells of Progeria Patients and Zmpste24^{-/-} Mice in Culture, and Partially Restores the Mechanisms for Repairing X-ray Irradiation-Induced DNA Lesions (FIGS. 10, 11, 12 and 13).

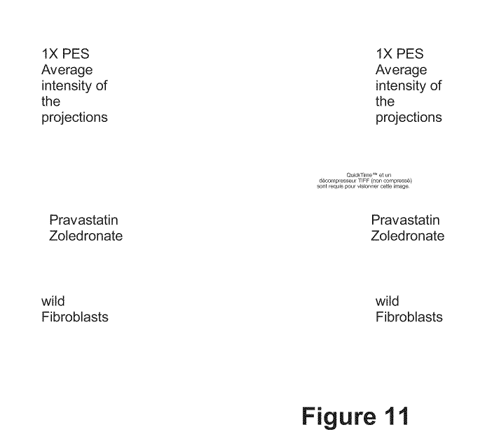

The pravastatin+zoledronate treatment causes the appearance of prelamin A in the nucleus of control cells (FIG. 10a), as in the nucleus of progeria patient cells, but with a marked improvement in the nuclear morphology of the latter (FIG. 10b). Quantitative analysis shows an increase in the nuclear anomalies in progeria patient cells with the number of passages, a number of anomalies which decreases under the effects of the pravastatin+zoledronate treatment (FIG. 10c). Observed under a confocal microscope, the cells of progeria patients contained aggregates of lamin A/C, deep invaginations of the nucleoplasmic face of the nuclear envelope in the nucleoplasm (nuclear reticulum) marked by anti-calreticulin antibodies (FIGS. 11a-f). These aggregates of lamin A/C were absent from control subject cells (FIGS. 10j-l) and disappeared from the cells of progeria patients under the effects of the pravastatin+zoledronate treatment (FIGS. 10g-i). The location of lamin B1, a farnesylated constituent of the nuclear lamina, was modified under the effects of the treatment, which confirms that the treatment blocks prenylation of the lamins.

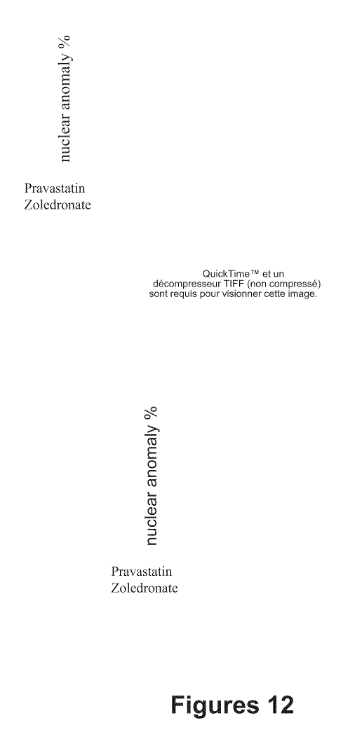

We have verified that the improvement in the shape of the nuclei as a result of the pravastatin+zoledronate treatment is indeed linked to the blocking of progerin prenylation, by incubating the cells with farnesol and/or geranylgeraniol. Supplementing the cells with farnesol and geranylgeraniol enables the cells to synthesize farnesylpyrophosphate and geranylgeranyl pyrophosphate and to therefore prenylate the progerin even in the presence of pravastatin+zoledronate (FIG. 10d). Farnesol abolishes the effect of the pravastatin+zoledronate treatment, which is another proof that the effect of the treatment is by way of inhibiting the synthesis of farnesylpyrophosphate. It is to be noted that geranylgeraniol also blocks the effect of the treatment, which proves that the geranylgeranylated form of progerin is also toxic to cells (FIG. 10d). The same effects were observed in the Zmpste24^{-/-} cells (FIG. 12a), which suggests that the data relating to progerin can be extended to prelamin A, the protein accumulated in the Zmpste24^{-/-} cells. Neither farnesol nor geranylgeraniol produces any effect on control fibroblasts, thereby eliminating the possibility of an artefact induced by these molecules (FIG. 12b).

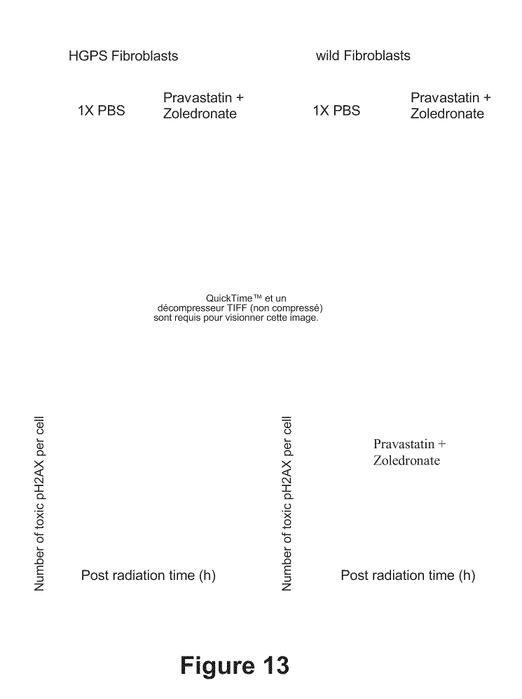

Finally, the pravastatin+zoledronate treatment causes a reduction in the number of foci of the phosphorylated histone H2AX, foci which are directly correlated with the number of unrepaired double-stranded breaks of the DNA (FIG. 13).

In conclusion, the data acquired in vitro shows that the pravastatin+zoledronate combination partially inhibits farnesylation and geranylgeranylation, and causes expected localization-related modifications of the lamina and redistribution, within the nucleoplasm, of the non-prenylated prelamin A and progerin in Zmpste24^{-/-} and progeria patient cells. Furthermore, the reduction in the quantity of farnesylated progerin in the lamina and the relocalisation thereof in the nucleoplasm explains the beneficial effects of the treatment on progeria patient cells.

Legend to FIG. 10: Synergistic effect of the pravastatin+zoledronate combination on the accumulation of prelamin A in control and progeria patient cells.

(a) Immunocytochemical detection of lamin A/C and prelamin A in normal human fibroblasts, which are untreated, treated with pravastatin (60 μM, 12 hours), with zoledronate (60 μM, 6 hours), alone or in combination. (b) Immunocytochemical detection of lamin A/C and of prelamin A in normal human and progeria patient fibroblasts treated for 24 hours with the pravastatin+zoledronate combination (1 μM each). (c) Quantitative analysis of the effect of the pravastatin+zoledronate treatment (1 μM each) on the nuclear morphology of progeria patient cells. The treated or untreated cells were immunolabelled with an anti-lamin A/C antibody at passages 8 (p8), 13 (p13) and 20 (p20). The white arrows show the abnormal nuclei. (d) Quantitative analysis of the effect of the pravastatin+zoledronate treatment (1 μM each) on the nuclear morphology of progeria patients in the presence of farnesol, geranylgeraniol or both compounds. Error bars=mean±standard error of the mean. Scale bar=10 μm.

Legend to FIG. 11: The pravastatin+zoledronate treatment corrects the nuclear morphology and induces partial relocalisation of the isoforms of lamin A/C and lamin B1 of the nuclear lamina in the nucleoplasm, in progeria patient cells.

(A) Colonisation of lamin A/C and of calreticulin in these treated or untreated progeria cells. Immunofluorescence and confocal microscopy (Leica TCS SP5, three-dimensional stack of 2048×2048 pixel images, increment size of 0.2 μm, average of 3 lines, accumulation of 3 images, zoom×1.7). The images a to c of each panel are projections of average intensity of 27 images of the stack and show the tubules of the nuclear reticulum labelled by the calreticulin in the progeria cells incubated with PBS. Images d to 1: isolated confocal sections of 0.2 μm in thickness. The pravastatin+zoledronate treatment corrects the form of the progeria cells, reduces the number of tubules of the nuclear reticulum (g) and the thickness of the nuclear lamina (h).

(B) Colocalisation of lamin B1 and calreticulin. The pravastatin+zoledronate treatment increases the nucleoplasmic labelling signal by lamin B1, which indicates that the farnesylation of this protein is partially inhibited. Scale bar=5 μm.

Legend to FIG. 12: The precursors of farnesylpyrophosphate and geranylgeranyl pyrophosphate abolish the effect of the pravastatin+zoledronate treatment in cells of Zmpste24^{-/-} mice in culture.

Quantification of the effect of pravastatin (1 μM) and zoledronate (1 μM) combined or uncombined on the nuclear morphology of Zmpste24^{-/-} mice (a) and control mice (b) cells in culture, in the presence of farnesol, geranylgeraniol or both molecules.

Farnesol and geranylgeraniol, either alone or in combination, abolish the effect of the pravastatin+zoledronate treatment on the nuclear morphology of Zmpste24^{-/-} cells.

Legend to FIG. 13: The pravastatin+zoledronate treatment partially corrects the repair anomalies of the double-stranded breaks of the DNA in progeria patient cells.

Control and progeria patient fibroblasts were incubated with the pravastatin+zoledronate mixture (1 μM each) or with PBS, and were irradiated with X-rays (2 Gy). Immunodetection of the phosphorylated histone H2AX foci detected 24 hours after irradiation, foci corresponding to the unrepaired double-stranded breaks (top images). DAPI nuclear labelling (bottom images).

Curves at the bottom: evolution in the number of phosphorylated histone H2AX foci in relation to time, after irradiation in control cells (solid square) and the progeria cells (empty circle), incubated with PBS or treated with pravastatin+zoledronate. Each curve represents the mean±standard error of the mean of at least 3 experiments.

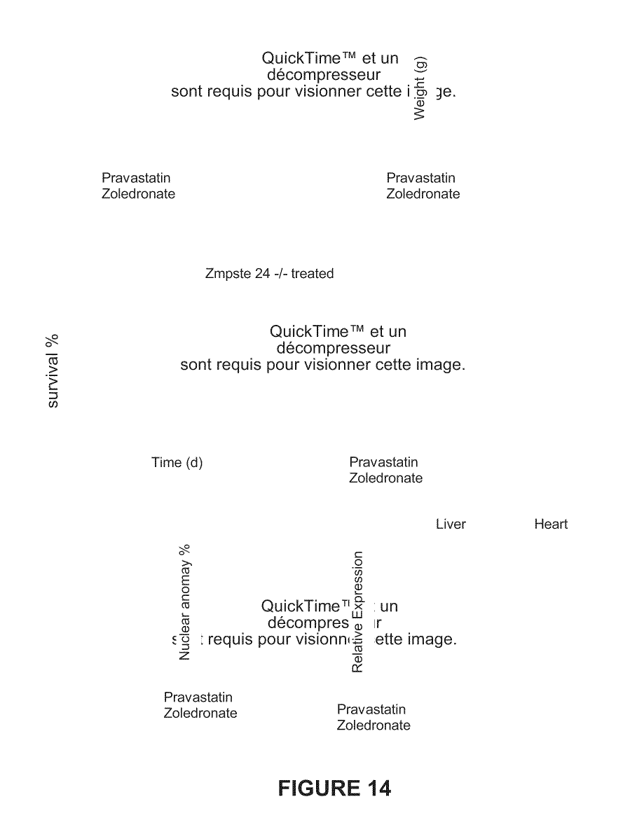

### The Treatment Combining Pravastatin+Zoledronate Improves the Progeroid Phenotype of Zmpste24^{-/-} Mice (FIGS. 14, 15 and 16):

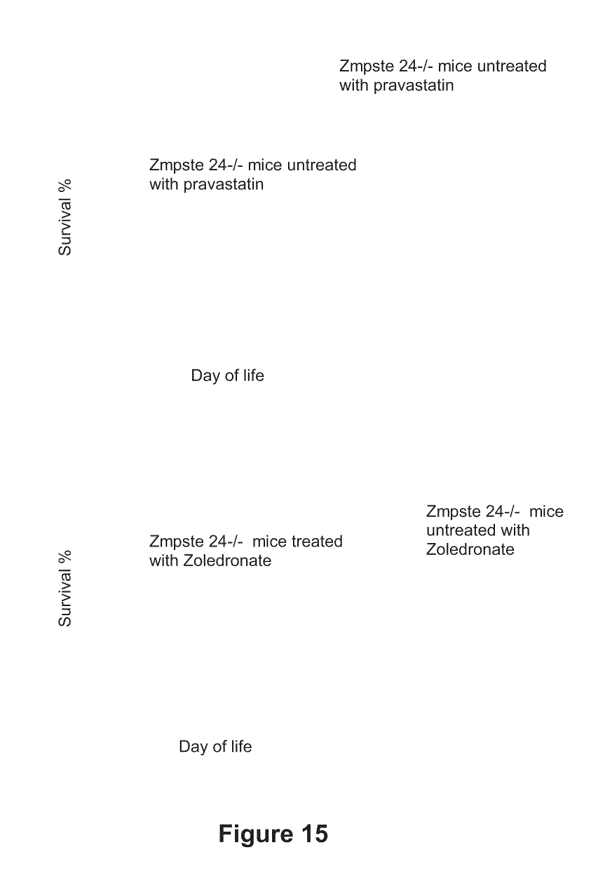

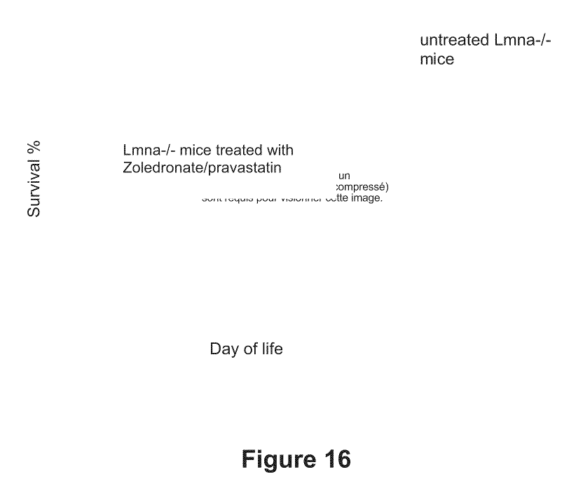

The Zmpste24^{-/-} mice and control mice were treated daily with pravastatin, zoledronate or the combination of both drugs, at a dose which had previously been shown to be non-toxic to mice. As already observed for progeria patient cells, each of the drugs separately, pravastatin or zoledronate, does not improve the lifespan of the Zmpste24^{-/-} mice (FIG. 15). On the other hand, the combination of the two drugs significantly improves the progeroid phenotype of the Zmpste24^{-/-} mice: the treatment improves weight gain, increases the amount of sub-cutaneous fat, reduces the degree of kyphosis and alopecia and increases life expectancy. The average survival value shifts from 101 to 179 days and the maximum value shifts from 151 to 222 days (P<0.001, FIG. 14c). It is to be noted that all of the phenotypic signs corrected by the treatment in mice are also characteristics of progeria in man. The combined treatment corrects the decrease in bone density, which is one of the characteristics of the Zmpste24^{-/-} mice and of patients afflicted with progeria or a related progeroid syndrome. Computerized microtomography of the bones shows an increase in bone mineralization and in the thickness of the tibial cortex in the treated mice (FIG. 14d). Furthermore, quantification of the nuclear morphological anomalies in the liver of treated Zmpste24^{+/+}, Zmpste24^{-/-} mice and treated Zmpste24^{-/-} mice shows that the pravastatin+zoledronate treatment normalises the form of the Zmpste24^{-/-} cell nuclei (FIG. 14e). The treatment further corrects the increased transcription of the target genes of the protein p53, an increase which has been described in the Zmpste24^{-/-} mouse cells (Varela et al., 2005) (FIG. 14f). Finally, we sought to find out if the treatment might have an effect on Lmna^{-/-} mice which are unable to accumulate prelamin A. The pravastatin+zoledronate treatment had no effect on the lifespan of these mice (FIG. 16), which is an additional proof that this treatment can only have an effect in mice which accumulate prelamin A in the nuclear envelope.

Legend to FIG. 14: The pravastatin+zoledronate treatment improves the progeroid phenotype of Zmpste24^{-/-} mice:

(a) Photographs representative of 3-month old Zmpste24^{+/+}, Zmpste24^{-/-} mice and Zmpste24^{-/-} mice treated with the pravastatin (100 mg/kg/day) and zoledronate (100 mg/kg/day) combination. Scale bar=1 cm. (b) Weight of 3-month old Zmpste24^{+/+} mice (n=12), Zmpste24^{-/-} mice (n=13) and treated Zmpste24^{-/-} mice (n=15). (c) Kaplan-Meier curves showing a significant increase in the lifespan of the treated Zmpste24^{-/-} mice (n=15) compared with that of the untreated mice (n=13). (d) Computerized microtomographic three-dimensional representation of the tibia of treated and untreated Zmpste24^{-/-} mice (top image). The bottom panel represents the relative bone volume (volume of the bone tissue/volume of the tibia) and the number of bone trabeculae in the untreated (n=6) and treated (n=5) Zmpste24^{-/-} mice. (e) Quantification of the nuclear anomalies of the hepatocytes of Zmpste24^{+/+}, Zmpste24^{-/-} mice and treated Zmpste24^{-/-} mice. The white arrows show the abnormal nuclei. Scale bar=10 μm. (f) Relative expression of the target genes of p53 in the liver and heart of Zmpste24^{+/+}, Zmpste24^{-/-} mice and treated Zmpste24^{-/-} mice, as analysed by quantitative RT-PCR. *P<0.05; ** P<0.01; *** P<0.))1. The error bars represent the mean ±standard error of the mean.

Legend to FIG. 15: Neither pravastatin alone nor zoledronate alone increases the lifespan of the Zmpste24^{-/-} mice:

Kaplan-Meier curves show that pravastatin alone (n=5) (a), and zoledronate alone (n=5) (b) do not correct the lifespan of the treated Zmpste24^{+/+}, Zmpste24^{-/-} mice and treated (empty diamond) and untreated (solid circles, n=11) Zmpste24^{-/-} mice.

Legend to FIG. 16: The pravastatin+zoledronate treatment does not correct the lifespan of Lmna^{-/-} mice.

Kaplan-Meier curves show that the lifespan of Lmna^{-/-} mice treated with pravastatin+zoledronate (n=12, empty diamond), compared with that of untreated mice (solid circles, n=11). The pravastatin+zoledronate treatment has no effect on lamin A/C-free mice.

### Summary/Conclusion/Outlook

Several human progeroid syndromes, including Hutchinson-Gilford progeria, are caused by the accumulation in the nuclear envelope of a deleted (progerin) or undeleted farnesylated form of prelamin A. Progerin is also produced over the course of physiological ageing. Previous studies conducted with cells of patients afflicted with progeria showed that the inhibitors of farnesyltransferase (FTI) improve the morphology of the nuclei, suggesting that these inhibitors might constitute a treatment for these devastating syndromes.

The inventors have shown here that prelamin A and progerin undergo an alternative prenylation via geranylgeranyltransferase, when the farnesyltransferase is inhibited, which might explain the low degree of effectiveness of the FTIs in improving the phenotype of murine models of these progeroid syndromes.

They also show that the combination of a statin and an aminobiphosphonate effectively inhibits both farnesylation and geranylgeranylation of the prelamin A and progerin, and significantly improves the ageing phenotype of mice in which the gene coding for the Zmpste24 metalloprotease was inactivated, which is involved in the maturation of prelamin A. The improvement of the phenotype includes that of the growth and weight curve, lipodystrophy, hair loss and bone anomalies.

Furthermore, the lifespan of these mice is increased substantially.

This data opens up a new therapeutic approach to human progeroid syndromes involving an accumulation of prenylated proteins in the nuclear envelope.

The pravastatin+aminobiphosphonate treatment is in the process of being used in Marseille for the next 3 years on children afflicted with progeria, within the framework of a European therapeutic trial (15 children) placed under the responsibility of Prof. Nicolas Levy, and funded by the Ministry of Health (PHRC 2008) and the French Muscular Dystrophy Association (AFM), and which has received authorization from the AFSSAPS and CCP Sud-Méditerranée.

The same treatment will soon be provided in Rome, under the responsibility of Prof. Giuseppe Novelli, to patients afflicted with acromandibular dysplasia, another progeroid syndrome involving an accumulation of farnesylated prelamin A.

### LIST OF REFERENCES

(1) Basso A D, Kirschmeier P, Bishop W R. Farnesyl transferase inhibitors. J Lipid Res 47:15-31, 2006.
(2) Biamonti G, Giacca M, Perini G, Contreas G, Zentilin L, Weighardt F, Guerra M, Della Valle G, Saccone S, Riva S et al. The gene for a novel human lamin maps at a highly transcribed locus of chromosome 19 which replicates at the onset of S-phase. Mol Cell Biol 12:3499-3506, 1992.
(3) Bishop W R, Kirschmeier P, Baun C. Farnesyl transferase inhibitors:

mechanism of action, translational studies and clinical evaluation. Cancer Biol Ther 2:S96-104, 2003.
(4) Broers J L, Hutchinson C J, Ramaekers F C. Laminopathies. J Pathol 204:478-488, 2004.
(5) Broers J L V, Ramaekers F C S, Bonne G, Ben Yaou R, Hutchinson C J. Nuclear lamins: laminopathies and their role in premature aging. Physiol Rev 86:967-1008, 2006.
(6) Capell B C, Erdos M R, Madigan J P, Fiordalisi J J, Varga R, Conneely K N, Gordon L B, Der C J, Cox A D, Collins F S. Inhibiting farnesylation of progerin prevents the characteristic nuclear blebbing of Hutchinson-Gilford progeria syndrome. Proc Natl Acad Sci USA 102:12879-12884, 2005.
(7) De Sandre-Giovannoli A, Bernard R, Cau P, Navarro C, Amiel J, Boccaccio I, Lyonnet S, Stewart C L, Munnich A, Le Merrer M, Levy N. Lamin A truncation in Hutchinson-Gilford progeria. Science 300:2055, 2003.
(8) Demyanets S, Kaun C, Pfaffenberger S, Philipp J. Hohensinner P J, Rega G, Pammer J, Maurer G, Huber K, Wojta J. Hydroxymethylglutaryl-coenzyme A reductase inhibitors induce apoptosis in human cardiac myocytes in vitro. Biochem Pharmacol 71:1324-1330, 2006.
(9) Duque G, Rivas D. Age-related changes in Lamin A/C expression in the osteoarticular system: laminopathies as a potentiel new aging mechanism. Mech Aging Dev 127:378-383, 2006.
(10) Efuet E T, Keyomarsi K. Farnesyl and geranylgeranyl transferase inhibitors induce G1 arrest by targeting the proteasome. Cancer Res 66:1040-1051, 2006.
(11) Eriksson M, Brown W T, Gordon L B, Glynn M W, Singer J, Scott L, Erdos M R, Robbins C M, Moses T Y, Berglund P, Dutra A, Pak E, Durkin S, Csoka A B, Boehnke M, Glover T W, Collins F S. Recurrent de novo point mutation in lamin A cause Hutchinson-Gilford progeria syndrome. Nature 423:293-298, 2003.
(12) Evans M, Rees A. The myotoxicity of statins. Cur Op Lipid, 13:415-420, 2002.
(13) Flint O P, Masters B A, Gregg R E, Durham S K. HMG CoA reductase inhibitor-induced myotoxicity: pravastatin and lovastatin inhibit the geranylgeranylation of low-molecular-weight proteins in neonatal rat muscle cell culture. Tox Appl Pharmacol 145:99-110, 1997.
(14) Fong L G, Frost D, Meta M, Qiao X, Yang S H, Coffinier C, Young S G. A protein farnesyltransferase inhibitor ameliorates disease in a mouse model of progeria. Science,311 : 1621-1623, 2006.
(15) Fong L G, Ng J K, Lammerding J, Vickers T A, Meta M, Coté N, Gavino B, Qiao X, Chang S Y, Young S R, Yang S H, Stewart C L, Lee R T, Bennett C F, Bergo M O, Young S G. Prelamin A and Lamin A appear to be dispensable in the nuclear lamina. J Clin Invest 116:743-752, 2006.
(16) Fong L G, Ng J K, Meta M, Cote N, Yang S H, Stewart C L, Sullivan T, Burghardt A, Majumdar S, Reue K, Bergo M O, Young S G. Heterozygosity for Lmna deficiency eliminates the progeria-like phenotypes in Zmpste24-deficient mice. Proc Natl Acad Sci USA 101:18111-18116, 2004.
(17) Glynn M W, Glover T W. Incomplete processing of mutant lamin A in Hutchinson-Gilford progeria leads to nuclear abnormalities, which are reversed by farnesyltransferase inhibition. Hum Mol Genet 14:2959-2969, 2005.
(18) Goldman R D, Shumaker D K, Erdos M R, Eriksson M, Goldman A E, Gordon L B, Gruenbaum Y, Khuon S, Mendez M, Varga R, Collins F S. Accumulation of mutant lamin A causes progressive changes in nuclear architecture in Hutchinson-Gilford progeria syndrome. Proc Natl Acad Sci USA 101:8963-8968, 2004.
(19) Gruenbaum Y, Margalit A, Goldman R D, Shumaker D K, Wilson K L. The nuclear lamina comes of age. Nat Mol Cell Biol 6:21-31, 2005.
(20) Hampton R, Dimster-Denk D, Rine J. The biology of HMG-CoA reductase: the pros of contra-regulation. Trends Biochem Sci 21:140-145, 1996.
(21) Harborth J, Elbashir S M, Bechert K, Tuschl T, Weber K. Identification of essential genes in cultured mammalian cells using small interfering RNAs. J Cell Sci 114:4557-4565, 2001.
(22) Hegele R A, Cao H, Liu D M, Costain G A, Charlton-Menys V, Rodger N W, Durrington P N. Sequencing of reannotated LMNB2 gene reveals novel mutations in patients with acquired partial lipodystrophy. Am J Hum Genet 79:383-389, 2006.
(23) Hildebrand T, Ruegsegger P. A new method for the model independent assessment of thickness in three dimensional images. J Microsc 185:67-75, 1997.
(24) Hoffmann G F, Charpentier C, Mayatepek E, Mancini J, Leichsenring M, Gibson K M, Divry P, Hrebicek M, Lehnert W, Sartor K. Clinical and biochemical phenotype in 11 patients with mevalonic aciduria. Pediatrics 91:915-921, 1993.
(25) Huang S, Chen L, Libina N, Janes J, Martin G M, Campisi J, Oshima J._{—}Correction of cellular phenotypes of Hutchinson-Gilford Progeria cells by RNA interference. Hum Genet. 2005 Oct. 6;: 1-7
(26) Hutchinson C J, Worman H J. A-type lamins: guardians of the soma? Nat Cell Biol 6:1062-1067, 2004.
(27) Kusuyama T, Omura T, Nishiya D, Enomoto S, Matsumoto R, Murata T, Takeuchi K, Yoshikawa J, Yoshiyama M. The effects of HMG-CoA reductase inhibitor on vascular progenitor cells. J Pharmacol Sci 1001:344-349, 2006.
(28) Leung K F, Baron R, Seabra M C. Geranylgeranylation of Rab GTPases, J Lipid Res 47:467-475, 2006.
(29) Levy N, Cau P. Anomalies du noyau et maladies. Pour la Science 313 :2-7, 2003.
(30) Lin F, Worman H J. Structural organization of the human gene (LMNB1) encoding nuclear lamin B1. Genomics 27:230-236, 1995.
(31) Lin F, Worman H J. Structural organization of the human gene encoding nuclear lamin A and nuclear lamin C. J Bid Chem 268:16321-16326, 1993.
(32) Liu Y, Wang Y, Rusinol A E, Sinensky M S, Liu J, Shell S M, Zou Y. Involvement of xeroderma pigmentosum group A (XPA) in progeria arising from defective maturation of prelamin A. FASEB J, 2007 Sep. 11:
(33) Mattout A, Dechal T, Adam S A, Goldman R D, Gruenbaum Y. Nuclear lambs, diseases and aging. Cur Op Cell Biol 18:335-341, 2006.
(34) McClintock D, Rather D. Lokuge M, Owens D M, Gordon L B, Collins F S, Djhabali K. The mutatnt form of lamin A that causes Hutchinson-Gifford progeria is a biomarker of cellular aging in human skin. PloS One, 2007, 2, e1269
(35) Navarro C L, Cadinanos J, De Sandre-Giovannoli A, Bernard R, Courrier S, Boccaccio I, Boyer A, Kleijer W J, Wagner A, Giuliano F. Beemer F A. Freije J M, Cau P. Hennekam R C, Lopez-Otin C, Badens C, Levy N. Loss of ZMPSTE24 (FACE-1) causes autosomal recessive restrictive dermopathy and accumulation of Lamin A precursors. Hum Mol Genet 14:1503-1513, 2005.
(36) Navarro C L, De Sandre-Giovannoli A, Bernard R, Boccaccio I, Boyer A, Genevieve D, Hadj-Rabia S, Gaudy-Marqueste C, Smitt H S, Vabres P, Faivre L, Verloes A, Van Essen T, Flori E, Hennekam R, Beemer F A, Laurent N, Le Mercer M, Cau P, Levy N. Lamin A and ZMPSTE24 (FACE-1) defects cause nuclear disorganization and identify restrictive dermopathy as a lethal neonatal laminopathy. Hum Mol Genet 13:2493-2503, 2004.
(37) Padiath O S, Saigoh K, Schiffmann R, Asahara H, Yamada T, Koeppen A. Hogan K, Ptacek L J, Fu Y H. Lamin B1 duplications cause autosomal dominant leukodystrophy. Nature Genet 38:1114-1123, 2006.
(38) Pendas A M, Zhou Z, Cadinanos J, Freije J M, Wang J, Hultenby K, Astudillo A, Wernerson A, Rodriguez F, Tryggvason K. Lopez-Otin C. Defective prelamin A processing and muscular and adipocyte alterations in Zmpste24 metalloproteinase-deficient mice. Nat Genet 31:94-99, 2002.
(39) Reid T S, Terry K L, Casey P J, Beese L S. Crystallographic analysis of CaaX prenyltransferases complexed with substrates defines rules of protein substrate selectivity. J Mol Biol 343:417-433, 2004.
(40) Scaffidi P, Misteli T. Lamin A-dependent nuclear defects in human aging. Sciencexpress, 27 avril 2006.
(41) Scaffidi P, Misteli T. Reversal of the cellular phenotype in the premature aging disease Hutchinson-Gifford progeria syndrome. Nat Med 11:440-445, 2005.
(42) Scaffidi P, Misteli T. Reversal of the cellular phenotype in the premature aging disease Hutchinson-Gilford progeria syndrome. Nature Med 11:440-445, 2005.
(43) Shelton K R, Egle P M, Cochran D L. Nuclear envelope proteins. identification of lamin B subtypes. Biochem Biophys Res Comm 103:975-981, 1981.
(44) Shumaker D K, Kuczmarski E R, Goldman R D. The nucleoskeleton: lamins an actin am major players in essential nuclear functions. Curr Op Cell Biol 15:358-366, 2003.
(45) Stewart C, Burke B. Teratocarcinoma stem cells and early mouse embryos contain only a single major lamin polypeptide closely resembling lamin B. Cell 51:383-392, 1987.
(46) Takedaa M, Noshiroa R, Onozatob M L, Tojob A, Hasannejada H, Huangc X L, Narikawac S, Endoua H. Evidence for a role of human organic anion transporters in the muscular side effects of HMG-CoA reductase inhibitors. Eur J Pharm 483 133-138 2004.
(47) Toth J I, Yang S H, Qiao X. Beigneux A P, Gelb M H, Moulson C L, Miner J H, Young S G, Fong L G. Blocking protein farnesyltransferase improves nuclear shape in fibroblasts from humans with progeroid syndromes. Proc Natl Aced Sci USA 102:12873-12878, 2005.
(48) Tsai M Y, Wang S. Heidinger J M, Shumaker D K, Adam S A, Goldman R D, Zheng Y. A mitotic lamin B matrix induced by RanGTP required for spindle assembly. Science 311:1887-1893, 2006.
(49) Varela I, Cadinanos J, Pendas A M, Gutierrez-Fernandez A, Folgueras A R, Sanchez L M, Zhou Z, Rodriguez F J, Stewart C L, Vega J A, Tryggvason K, Freije J M, Lopez-Otin C. Accelerated ageing in mice deficient in Zmpste24 protease is linked to p53 signalling activation. Nature 437:564-568, 2005.
(50) Vergnes L, Peterty M, Bergo M O, Young S G, Reue K. Lamin B1 is required for mouse development and nuclear integrity. Proc Natl Acad Sci USA 101:10428-10433, 2004.
(51) Winter-Vann A M, Casey P J, Post-prenylation-processing enzymes as new targets in oncogenesis. Nat Rev Cancer 5:405-412, 2005.
(52) Wydner K L, McNeil J A, Lin F, Worman H J, Lawrence J B.

Chromosomal assignment of human nuclear envelope protein genes LMNA, LMNB1 and LBR by fluorescence in situ hybridization. Genomics 32:474-478, 1996.
(53) Young S G, Meta M, Yang S H, Fang L G. Prelamin A farnesylation and progeroid syndromes. J Biol Chem 281:39741-39745, 2006.
(54) Zastrow M S, Vicek S, Wilson K L. Proteins that bind A-type lamins: integrating isolated clues. J Cell Sci 117:979-987, 2004,
(55) Varela, I. Pereira, S. Ugalde, A. P. Navarro, C. L. Suarez, M. F. Cau, P. Cadinanos, J. Osorio, F. G. Foray, N. Cobo, J. de Carlos, F. Levy, N. Free, J. M. Lopez-Otin, C. Combined treatment with statins and aminobisphosphonates extends longevity in a mouse model of human premature aging. Nature Medicine 14; 767-772, 2008
(56) Sullivan, T. Escalante-Alcalde, D, Bhatt, H. Anver, M. Bhat, N. Nagashima, K. Stewart, C. L. Burke, B. Loss of A-type lamin expression compromises nuclear envelope integrity leading to muscular dystrophy J. Cell Biol. 147: 913-920, 1999

Key to the figures
FIG. 1

| | |
|---|---|
| Prélamine A | Prelamin A |
| GAPD | GAPD |

FIG. 2

| | |
|---|---|
| Inhibiteur HMG CoA | HMG CoA reductase |
| reductase + inhibteur | inhibitor + farnesyl-PP |
| farnésyl-PP synthase | synthase inhibitor |
| PBS1X | 1 X PBS |

FIG. 3

| | |
|---|---|
| % Noyaux anormaux | % Abnormal nuclei |
| % Prélamine A | % Prelamin A |

FIG. 4

| | |
|---|---|
| Cellules HGPS traitées | Treated HPGS cells |
| Placebo | Placebo |

FIG. 5

| | |
|---|---|
| Index mitotique | Mitotic index |
| Données non disponibles | Unavailable data |
| agés | aged |
| Aucun | None |
| jeunes | young |
| conditions | conditions |

FIG. 7

| | |
|---|---|
| % intensité | % intensity |

FIG. 8

| | |
|---|---|
| % intensité | % intensity |

FIG. 9

| | |
|---|---|
| Poucentage d'intensité | Percentage of intensity |

FIG. 10

| | |
|---|---|
| Lamine A/C | Lamin A/Cq |
| prélamine | Prelamin |
| superposition | Overlay |
| Pravastatine | Pravastatin |
| Zolédronate | Zoledronate |
| Pravastatine | Pravastatin |
| Zolédronate | Zoledronate |
| Non traité | Untreated |
| Fibroblastes contrôles | Controle fibroblasts |
| Traité | Treated |
| Anomalie nucléaire | Nuclear anomaly |

FIG. 11

| | |
|---|---|
| PES 1X | 1 X PES |
| Intensité moyenne des | Average intensity of the |
| projections | projections |
| Pravastatine | Pravastatin |
| Zoledronate | Zoledronate |
| Fibroblastes sauvage | Wild fibroblasts |

FIG. 12

| | |
|---|---|
| Anomalie nucléaire | Nuclear anomaly |
| Pravastatine | Pravastatin |
| Zoledronate | Zoledronate |

FIG. 13

| | |
|---|---|
| Fibroblastes HGPS | HGPS fibroblasts |
| Fibroblastes sauvages | Wild fibroblasts |
| PBS 1X | 1 X PBS |
| Pravastatine + | Pravastatin + |
| Zolédronate | Zoledronate |
| Nombre de pH2AX toxique | Number of toxic pH2AX per |
| par cellule | cell |
| Temps post radiation (h) | Post-radiation time (h) |

FIG. 14

| | |
|---|---|
| Pravastatine | Pravastatin |
| Zolédronate | Zoledronate |
| Poids (g) | Weight (g) |
| traité | treated |
| % de survie | Survival % |
| Temps (d) | Time (d) |
| Anomalie nucléaire % | Nuclear anomaly % |
| Expression relative | Relative expression |

FIG. 15

| | |
|---|---|
| Souris Zmpste 24-/- non | Zmpste 24-/- mice |
| traitées avec la | untreated with pravastatin |
| pravastatine | |
| Souris Zmpste 24-/- | Zmpste 24-/- mice treated |
| traitées avec la | with pravastatin |
| pravastatine | |
| % de survie | Survival % |
| Jour de vie | Day of life |
| Souris Zmpste 24-/- non | Zmpste 24-/- mice |
| traitées avec le | untreated with Zoledronate |
| Zolédronate | |

FIG. 16

| | |
|---|---|
| Souris Lmna-/- non | Untreated Lmna-/- mice |
| traitées | |
| Souris Lmna-/- treated | Lmna-/- mice treated with |
| with | Zoledronate/pravastatin |
| Zolédronate/pravastatine | |
| Jour de vie | Day of life |
| % de survie | Survival % |

1. A composition for manufacturing a cosmetic and/or dermatological product intended for the treatment of skin and/or hair disorders, said composition comprising:
at least one inhibitor of hydroxymethylglutaryl-coenzyme A (HMG-CoA) reductase, and
at least one inhibitor of farnesyl pyrophosphate synthase.

2. The composition of claim 1, wherein the HMG-CoA reductase inhibitor is a molecule of the family of statins or one of the physiologically acceptable salts thereof.

3. The composition of claim 2, wherein the HMG-CoA reductase inhibitor is a water-soluble statin.

4. The composition of claim 2, wherein the HMG-CoA reductase inhibitor is selected from the group consisting of atorvastatin, simvastatin, pravastatin, rivastatin, mevastatin (or compactin), fluindostatin, velostatin, fluvastatin, dalvastatin, cerivastatin, pentostatin, rosuvastatin, lovastatin, pitavastatin, and one of the physiologically acceptable salts thereof.

5. The composition of 1, wherein the farnesylpyrophosphate synthase inhibitor is selected from the group consisting of a molecule of the family of aminobiphosphonates (NBPs) and one of the physiologically acceptable salts thereof.

6. The composition of claim 5, wherein the aminobiphosphonate is selected from the group consisting of:
alendronic acid or the ionic form thereof,
clodronic acid or the ionic form thereof,
etidronic acid or the ionic form thereof,
ibandronic acid or the ionic form thereof,
medronic acid or the ionic form thereof,
neridronic acid or the ionic form thereof,
olpadronic acid or the ionic form thereof,
pamidronic acid or the ionic form thereof,
risedronic acid or the ionic form thereof,
tiludronic acid or the ionic form thereof,
zoledronic acid or the ionic form thereof,
4-N,N-dimethylaminomethane diphosphonic acid or the ionic form thereof, and
α-amino-(4-hydroxybenzylidene)diphosphonate.

7. The composition of claim 1, wherein the farnesylpyrophosphate synthase inhibitor is zoledronic acid or the ionic form thereof, zoledronate.

8. The composition of claim 1, wherein the quantity of farnesylpyrophosphate synthase inhibitor is from 0.001 to 0.050 percent by weight of the cosmetic and/or dermatological composition.

9. The composition of claim 1, wherein the quantity of HMG-CoA reductase inhibitor is from 0.010 to 0.100 percent by weight of the cosmetic and/or dermatological composition.

10. The composition of claim 1, wherein the sum of the quantity of HMG-CoA reductase inhibitor and quantity of farnesylpyrophosphate synthase inhibitor is between 0.011 and 0.150 percent by weight of the cosmetic and/or dermatological composition.

11. The composition of claim 1, further comprising at least one of the compounds selected from the group consisting of surfactant, thickening agent, gelling agent, preservative, humectant, emulsifier, perfume, silicone, chelating agent, antioxidant, cosmetic dye, fungicide, antibacterial agent, film-forming agent, stabilizer, buffering agent, UV filter, binder and emulsion stabilizer.

12. The composition of claim 1, wherein the composition is a composition for topical, transdermal or transcutaneous use or a composition for oral administration.

13. The composition of claim 1, wherein the composition is in the form of a cream, aerosol, gel, ointment, powder, foam, milk, food supplement or orally administrable product.

14. The composition of claim 1, wherein the skin and/or hair disorder is chosen from the group including skin ageing, premature skin ageing, premature myolipocutaneous ageing, progeria, lipodystrophy and/or restrictive dermopathy.

15. A method for the treatment of skin or hair disorder comprising the step of administering to an individual in need thereof at least one inhibitor of hydroxymethylglutaryl-coenzyme A (HMG-CoA) reductase, and at least one inhibitor of farnesyl pyrophosphate synthase.

16. The method of claim 15, wherein the HMG-CoA reductase inhibitor is selected from the group consisting of atorvastatin, simvastatin, pravastatin, rivastatin, mevastatin (or compactin), fluindostatin, velostatin, fluvastatin, dalvastatin, cerivastatin, pentostatin, rosuvastatin, lovastatin, pitavastatin, or one of the physiologically acceptable salts thereof.

17. The method of claim 15, wherein the aminobiphosphonate is selected from the group consisting of:
alendronic acid or the ionic form thereof,
clodronic acid or the ionic form thereof,
etidronic acid or the ionic form thereof,
ibandronic acid or the ionic form thereof,
medronic acid or the ionic form thereof,
neridronic acid or the ionic form thereof,
olpadronic acid or the ionic form thereof,
pamidronic acid or the ionic form thereof,
risedronic acid or the ionic form thereof,
tiludronic acid or the ionic form thereof,
zoledronic acid or the ionic form thereof,
4-N,N-dimethylaminomethane diphosphonic acid or the ionic form thereof, and
α-amino-(4-hydroxybenzylidene)diphosphonate.

18. The method of claim 15, wherein the administrating step is joint, simultaneous, concomitant, or successive.

19. The method of claim 15, wherein the at least one inhibitor of hydroxymethylglutaryl-coenzyme A (HMG-CoA) reductase and the at least one inhibitor of farnesyl pyrophosphate synthase are included in a cosmetic product.

20. A method for making a cosmetic and/or dermatological product comprising the step of combining at least one inhibitor of hydroxymethylglutaryl-coenzyme A (HMG-CoA) reductase and at least one inhibitor of farnesyl pyrophosphate synthase.
